SEQUENCE LISTING (38 sequences)

SEQ ID NO: 1 - DNA, 22 bp, Artificial Sequence
  Thy1_Forward Primer
gaaggtcctctacttatccgcc

SEQ ID NO: 2 - DNA, 22 bp, Artificial Sequence
  Thy1_Reverse Primer
tgatgccctcacacttgaccag

SEQ ID NO: 3 - DNA, 22 bp, Artificial Sequence
  Snail1_Forward Primer
tgccctcaagatgcacatccga

SEQ ID NO: 4 - DNA, 21 bp, Artificial Sequence
  Snail1_Reverse Primer
gggacaggagaagggcttctc

SEQ ID NO: 5 - DNA, 22 bp, Artificial Sequence
  Snail2_Forward Primer
atctgcggcaaggcgttttcca

SEQ ID NO: 6 - DNA, 22 bp, Artificial Sequence
  Snail2_Reverse Primer
gagccctcagatttgacctgtc

SEQ ID NO: 7 - DNA, 22 bp, Artificial Sequence
  CD44_Forward Primer
ccagaaggaacagtggtttggc

SEQ ID NO: 8 - DNA, 22 bp, Artificial Sequence
  CD44_Reverse Primer
actgtcctctgggcttggtgtt

SEQ ID NO: 9 - DNA, 22 bp, Artificial Sequence
  Alp_Forward Primer
cctgatggagatgacagaggct

SEQ ID NO: 10 - DNA, 22 bp, Artificial Sequence
  Alp_Reverse Primer
tcagtgagtgcctggtaagcca

SEQ ID NO: 11 - DNA, 22 bp, Artificial Sequence
  beta-catenin_Forward Primer
cacaagcagagtgctgaaggtg

SEQ ID NO: 12 - DNA, 23 bp, Artificial Sequence
  beta-catenin_Reverse Primer
gattcctgagagtccaaagacag

SEQ ID NO: 13 - DNA, 22 bp, Artificial Sequence
  Nanog_Forward Primer
ctccaacatcctgaacctcagc

SEQ ID NO: 14 - DNA, 23 bp, Artificial Sequence
  Nanog_Reverse Primer
cgtcacaccattgctattcttcg

SEQ ID NO: 15 - DNA, 22 bp, Artificial Sequence
  Lin28A_Forward Primer
ccagtggatgtctttgtgcacc

SEQ ID NO: 16 - DNA, 22 bp, Artificial Sequence
  Lin28A_Reverse Primer
gtgacacggatggattccagac

SEQ ID NO: 17 - DNA, 22 bp, Artificial Sequence
  Sall4_Forward Primer
gaaaccacatccttccaggcac

SEQ ID NO: 18 - DNA, 22 bp, Artificial Sequence
  Sall4_Reverse Primer
gataaacgtggaagggagactg

SEQ ID NO: 19 - DNA, 22 bp, Artificial Sequence
  E-cadherin_Forward Primer
cctcctgaaaagagagtggaag

SEQ ID NO: 20 - DNA, 22 bp, Artificial Sequence
  E-cadherin_Reverse Primer
tggcagtgtctctccaaatccg

SEQ ID NO: 21 - DNA, 22 bp, Artificial Sequence
  EpCAM_Forward Primer
gccagtgtacttcagttggtgc

SEQ ID NO: 22 - DNA, 23 bp, Artificial Sequence
  EpCAM_Reverse Primer
cccttcaggttttgctcttctcc

SEQ ID NO: 23 - DNA, 22 bp, Artificial Sequence
  OCT3/4 Forward Primer
cctgaagcagaagaggatcacc

SEQ ID NO: 24 - DNA, 22 bp, Artificial Sequence
  OCT3/4 Reverse Primer
aaagcggcagatggtcgtttgg

SEQ ID NO: 25 - DNA, 22 bp, Artificial Sequence
  SOX2_Forward Primer
gctacagcatgatgcaggacca

SEQ ID NO: 26 - DNA, 22 bp, Artificial Sequence
  SOX2_Reverse Primer
tctgcgagctggtcatggagtt

SEQ ID NO: 27 - DNA, 22 bp, Artificial Sequence
  Dppa4_Forward Primer
ctccacagagaagtcgagggaa

SEQ ID NO: 28 - DNA, 22 bp, Artificial Sequence
  Dppa4_Reverse Primer
ggttgtcagtgtgctctgcctt

SEQ ID NO: 29 - DNA, 22 bp, Artificial Sequence
  Klf4_Forward Primer
catctcaaggcacacctgcgaa

SEQ ID NO: 30 - DNA, 22 bp, Artificial Sequence
  Klf4_Reverse Primer
tcggtcgcatttttggcactgg

SEQ ID NO: 31 - DNA, 22 bp, Artificial Sequence
  GAPDH_Forward Primer
gtctcctctgacttcaacagcg

SEQ ID NO: 32 - DNA, 22 bp, Artificial Sequence
  GAPDH_Reverse Primer
accaccctgttgctgtagccaa

SEQ ID NO: 33 - DNA, 22 bp, Artificial Sequence
  Nodal_Forward Primer
tggcgtacatgctgagcctcta

SEQ ID NO: 34 - DNA, 22 bp, Artificial Sequence
  Nodal_Reverse Primer
cctcttgttggctcaggaagga

SEQ ID NO: 35 - DNA, 22 bp, Artificial Sequence
  GDF3_Forward Primer
gtctcccgagacttatgctacg

SEQ ID NO: 36 - DNA, 22 bp, Artificial Sequence
  GDF3_Reverse Primer
agtagaggagcttctgcaggca

SEQ ID NO: 37 - DNA, 22 bp, Artificial Sequence
  DNMT3B_Forward Primer
taacaacggcaaagaccgaggg

SEQ ID NO: 38 - DNA, 22 bp, Artificial Sequence
  DNMT3B_Reverse Primer
tcctgccacaagacaaacagcc

(19) United States
(12) Patent Application Publication
(10) Pub. No.: US 2024/0360420 A1
(43) Pub. Date: Oct. 31, 2024

(54) Reprogramming Somatic Cells on Microcarriers

(71) Applicant: AGENCY FOR SCIENCE, TECHNOLOGY AND RESEARCH, Singapore (SG)

(72) Inventors: Steve Kah-Weng Oh, Singapore (SG); Shaul Reuveny, Singapore (SG); Tin Lun Lam, Singapore (SG); Jonathan Yuin Han Loh, Singapore (SG)

(21) Appl. No.: 18/287,710

(22) PCT Filed: Apr. 21, 2022
(86) PCT No.: PCT/SG2022/050240

§ 371 Date: Oct. 20, 2023

(30) Foreign Application Priority Data
Apr. 23, 2021 (SG) 10202104162P

(51) Int. Cl. C12N 5/074; C12N 15/86

(52) U.S. Cl. CPC C12N 5/0696; C12N 15/86; C12N 2506/1307; C12N 2531/00; C12N 2533/30; C12N 2533/50; C12N 2760/18843

(57) ABSTRACT

Disclosed is a method of reprogramming somatic cells into induced pluripotent stem cells (iPSCs). Also disclosed is a method of producing, selecting, expanding, characterizing, and differentiating iPSCs. Further disclosed is a method of reprogramming somatic cells selected from the group consisting of fibroblasts IMR90, fibroblasts HFF-01, PBMC, CD3+ T cells and CD34+ hematopoietic stem cells (HSCs) into iPSCs.

### CROSS-REFERENCE TO RELATED APPLICATIONS

This application claims the benefit of priority of Singapore application Ser. No. 10202104162P, filed 23 Apr. 2021, the contents of it being hereby incorporated by reference in its entirety for all purposes.

### TECHNICAL FIELD

The present invention generally relates to a method of reprogramming somatic cells. In particular, the present invention relates to a method of reprogramming somatic cells on microcarriers into induced pluripotent stem cells.

### BACKGROUND

Human induced pluripotent stem cells (iPSCs) are derived from adult somatic cells by the introduction of genes that encode pluripotent behavior. Past studies have demonstrated that iPSCs resemble embryonic stem cells (ESCs) morphologically; they express similar cell surface and pluripotency markers, have a normal karyotype, express telomerase, and demonstrate multi-lineage differentiation potential in both embryoid bodies and teratomas. Since then, the field has expanded to generate iPSCs using vastly different approaches. It has been reported that iPSC derivation can be achieved using different means of transduction, such as adenoviruses, lentiviruses, Sendai virus (SeV), and plasmids. SeV is a single-stranded non-integrative RNA virus which can replicate in the cytoplasm of infected cells. SeV-mediated reprogramming is the most used integration-free method of iPSC production available. It has been used for effectively reprogramming of fibroblasts and peripheral blood mononuclear cells to iPSCs, with mean reprogramming efficiency of about 0.007%.

Regardless of the approach, the manufacturing of iPSCs for therapeutic purposes relies on starting from somatic cell acquisition, cellular reprogramming, iPSC expansion, quality assurance, master/working cell banking followed by downstream directed differentiation to a relevant functional cell type. However, several technical hurdles must be overcome before iPSCs can be translated to industrial use for drug screening or clinical applications. One of these obstacles is the scalable and reproducible production of iPSCs in adequate quantities for their applications.

Conventional reprogramming approach, which employs static monolayer (MNL) culture, (as exemplified in FIG. 1(A)) typically involved overexpressing four reprogramming transcription factors (which may be selected from, for example, Oct3/4, Sox2, Klf4, Nanog, c-Myc, and LIN28) in somatic cells. Different methods have been used to introduce these factors, including integrative, excisable, non-integrative and DNA-free methods. After induction, the reprogrammed cells were plated on an extracellular matrix (ECM)-coated (such as laminin and vitronectin) tissue culture plate and allowed formation of colony. Generally, cell colonies will be observed 1-2 weeks after the plating. Individual colony is then manually picked for validation and further expansion. Following expansion, the newly-derived iPSCs lines were cryopreserved for further characterization and differentiation into functional cells (e.g. cardiomyocytes and neurons). Conventional reprogramming approach has several disadvantages, such as being labor-intensive, time-consuming for cell passaging, and requires cell dissociation prior to differentiation. In particular, (i) it is hard to automate and need manual work; (ii) it employs enzymatic dissociation for sub-culturing and monitoring; (iii) it gives rise to limited amount of clones due to the small area of tissue culture plates; (iv) it requires a cell bank to be generated at initial stage before evaluation of growth and ability to differentiate; and (v) only small amount of the cell banks can be used for the final target (e.g. cardiomyocytes). Importantly, the limited number of derived cells may be unable to support potential clinical applications. iPSC generation on conventional monolayer cultures typically takes 6-8 weeks, with varying degrees of efficiency depending on the method of reprogramming.

In order to increase the reprogramming efficiency and ultimately scale up the production of these cells, researchers have tried to use bioreactor suspension culture to induce pluripotency of mouse fibroblasts to mouse iPSCs in the form of cell aggregates. However, it is not yet clear whether the suspension culture approach will work well for human cells.

The reprogramming of somatic cells into iPSCs and the development of methods for directing stem cell differentiation into relevant cell types offers an unprecedented opportunity to study the cellular phenotypes that underlie disease. Yet, as described above, feeding, reprogramming, and picking iPSCs colonies is very labour-intensive. Moreover, there are many technical hurdles, including the need for robust and large-scale reprogramming of somatic cells towards a pluripotent state for cell therapy. Until now there is a deficiency in industrial processes for automated large-scale generation and expansion of iPSCs.

Thus, there is a need for a method of reprogramming somatic cell into an undifferentiated cell, such as iPSCs, that addresses, or at least ameliorates, one or more of the disadvantages described above. A high-throughput method that enables the large-scale production of iPSCs holds great promise for revolutionizing regenerative medicine.

### SUMMARY

In a first aspect, the present disclosure refers to a method of reprogramming somatic cells into induced pluripotent stem cells (iPSCs), comprising:
(a) seeding the somatic cells on a plurality of microcarriers (MCs) to form cell-MC aggregates; and
(b) transducing transcription factors into the somatic cells of the cell-MC aggregates;
wherein step (a) and step (b) are carried out under continuous agitation.

In a second aspect, the present disclosure refers to a method of producing, selecting, expanding, characterizing, and differentiating iPSCs, comprising:
carrying out steps (a)-(b) of the first aspect;
(c) immobilizing the cell-MC aggregates into a hydrogel;
(d) selecting the cell-MC aggregates showing fast cell growth or expressing pluripotency markers;
(e) expanding the selected cell-MC aggregates by adding fresh MCs to the selected cell-MC aggregates;
(f) characterising the cell-MC aggregates to determine whether the somatic cells on the MCs have been reprogrammed to iPSCs; and
(g) differentiating the cells of the cell-MC aggregates toward functional cells.

In a third aspect, the present disclosure refers to a method of reprogramming somatic cells selected from the group consisting of fibroblasts IMR90, fibroblasts HFF-01, PBMC, CD3+ T cells and CD34+ hematopoietic stem cells (HSCs) into induced pluripotent stem cells (iPSCs), comprising:
(a) seeding the somatic cells on a plurality of microcarriers (MCs) to form cell-MC aggregates;
(b) transducing transcription factors into the somatic cells of the cell-MC aggregates using Sendai virus;
(c) immobilizing the cell-MC aggregates into a hydrogel;
(d) selecting the cell-MC aggregates showing fast cell growth or expressing pluripotency markers;
(e) expanding the selected cell-MC aggregates by adding fresh MCs to the selected cell-MC aggregates; and
(f) characterising the cell-MC aggregates to determine whether the somatic cells on the MCs have been reprogrammed to iPSCs;
   wherein step (a) and step (b) are carried out under continuous agitation; and
   wherein the transcription factors comprise Oct4, Sox2, c-Myc, and Klf4.

### BRIEF DESCRIPTION OF DRAWINGS

The present disclosure will be better understood with reference to the detailed description when considered in conjunction with the non-limiting examples and the accompanying drawings, in which:

FIG. 1, comprising FIGS. 1(A) and 1(B), is a schematic diagram for somatic cell reprogramming, wherein FIG. 1(A) shows conventional monolayer (MNL) approach, and FIG. 1(B) shows ReprograMC approaches (Method A and Method B).

FIG. 2 shows the morphology of derived-iPSCs colonies reprogrammed on monolayer cultures 2 hours and on Day 7 post transduction, on Day 15 (when manually picked), on Day 23 (when expanded on laminin-coated tissue culture plates), and on Day 31 (when frozen down for banking). Scale bars, 200 μm.

FIG. 3, comprising FIGS. 3(A) and 3(B), shows the characterization of iPSC clones derived from fibroblasts on monolayer cultures, wherein FIG. 3(A) shows high expression of the pluripotent markers Tra-1-60 and Oct4 detected by FACS analysis, and FIG. 3(B) shows immunochemical staining of pluripotency marker (Tra-1-60) which further confirms the pluripotency of iPSC clones. Scale bars, 500 μm.

FIG. 4, comprising FIGS. 4(A), 4(B) and 4(C), shows the monitoring, screening and selection of transduced cell-MC aggregates, wherein FIG. 4(A) shows the representative images of transduced IMR90 cell attachment, embedded hydrogel cultures, and cell-MC selection with Tra-1-60 pluripotent antibody marker, FIG. 4(B) shows the representative images of transduced blood T-cell attachment, embedded hydrogel cultures, and cell-MC selection with Tra-1-60 pluripotent antibody marker, and FIG. 4(C) shows the representatives images of transduced CD34+HSCs attachment, embedded hydrogel cultures, and cell-MC selection with Tra-1-60 pluripotent antibody marker. Scale bars, 200 μm.

FIG. 5, comprising FIGS. 5(A), 5(B) and 5(C), shows cell-MC aggregate expansion, wherein FIG. 5(A) shows the representative images of IMR90 cell-MC aggregates expansion by sub-culturing from 96-well plate to 12-well plate, and then to 6-well plate (where more fresh MCs was added in each sub-culturing, twelve clones were selected and 3 representative clones are presented), FIG. 5(B) shows the representative images of blood T cell-MC aggregates expansion by sub-culturing from 96-well plate to 12-well plate, and then to 6-well plate (where more fresh MCs was added in each sub-culturing and only 2 representative clones were selected), and FIG. 5(C) shows the representative images of cord blood CD34+HSCs-MC aggregates expansion by sub-culturing from 96-well plate to 12-well plate, and then to 6-well plate (where more fresh MCs was added in each sub-culturing and only 2 representative clones were selected). Scale bars, 200 μm.

FIG. 6, comprising FIGS. 6(A), 6(B), 6(C), 6(D) and 6(E), shows the pluripotent markers expression of the derived iPSC-MC clones, wherein FIG. 6(A) shows the pluripotent markers expression of different HFF-01-derived iPSC-MC clones, FIG. 6(B) shows the pluripotent markers expression of IMR90-derived iPSC-MC clones, FIG. 6(C) shows the pluripotent markers expression of PBMC-derived iPSC-MC clones, FIG. 6(D) shows the pluripotent markers expression of CD3+ T-cells-derived iPSC-MC clones, and FIG. 6(E) shows the pluripotent markers expression of CD34-derived iPSC-MC clones, where all iPSC-MC clones exhibited high pluripotency Tra-1-60, Oct4 and SSEA-4.

FIG. 7, comprising FIGS. 7(A), 7(B), 7(C), 7(D) and 7(E), shows the real-time PCR data for a pluripotent marker gene and genes associated with the formation of the 3 germ layers from the in vitro differentiated representative aggregates, wherein FIG. 7(A) shows the real-time PCR data of HFF-01-derived iPSC-MC clones, FIG. 7(B) shows the real-time PCR data of IMR90-derived iPSC-MC clones, FIG. 7(C) shows the real-time PCR data of PBMC-derived iPSC-MC clones, FIG. 7(D) shows the real-time PCR data of CD3+ T-cells-derived iPSC-MC clones, and FIG. 7(E) shows the real-time PCR data of CD34-derived iPSC-MC clones. An increased expression of 7 lineage-specific genes associated with the endoderm (AFP and GATA6), mesoderm (Hand1 and Nkx2.5), and ectoderm (Pax6 and Sox1), and a decreased expression of pluripotent marker Oct4 illustrated that the iPSCs clones has the ability to differentiate into the 3 germ layers.

FIG. 8, comprising FIGS. 8(A), 8(B), 8(C), 8(D) and 8(E), shows the images of immunochemical staining for markers associated with the 3 germ layers, AFP (endoderm), SMA (mesoderm), and β-III tubulin (ectoderm) from the in vitro differentiated cells, wherein FIG. 8(A) shows the images of immunochemical staining of HFF-01-derived iPSC-MC clones (HR01 and HR02), FIG. 8(B) shows the images of immunochemical staining of IMR90-derived iPSC-MC clones (IR01 and IR02), FIG. 8(C) shows the images of immunochemical staining of PBMC-derived iPSC-MC clones (BR01 and BR02), FIG. 8(D) shows the images of immunochemical staining of CD3+ T-cells-derived iPSC-MC clones (TR01 and TR02), and FIG. 8(E) shows the images of immunochemical staining of CD34-derived iPSC-MC clones (CR01 and CR02). Scale bars: 200 μm. This data was corroborated with real-time PCR results (FIG. 7) for markers associated with the 3 embryonic germ layers of cells on laminin-coated MCs.

FIG. 9, comprising FIGS. 9(A) and 9(B), shows the differentiation of HFF-01-derived iPSC-MC and IMR90-derived iPSC-MC clones, wherein FIG. 9(A) shows (I) cardiomyocytes differentiation, and (II) erythroid differentiation of 12 HFF-01-derived iPSC-MC clones, and FIG. 9(B) shows (I) cardiomyocytes differentiation, and (II) erythroid differentiation of 12 IMR90-derived iPSC-MC clones. Cardiac differentiation efficiency was evaluated by the percentage of cTnT+ cells and erythroid differentiation was measured by the percentage of DRAQ5+ cells. Results show variability in differentiation efficiency within different clones. As concluded, cell line-to-cell line variation may occur even if they are derived from the same source. Higher number of iPSC clones generated from the MC based platform provides higher chance to find the best clone for cell differentiation.

FIG. 10, comprising FIGS. 10(A), 10(B) and 10(C), shows hematopoietic stem cells differentiation from IMR90-derived, T-cells-derived, and CD34+HSCs-derived iPSC-MC clone, wherein FIG. 10(A) shows mesoderm/primitive-streak marker (T-bra) at day 1 of differentiation, FIG. 10(B) shows hematopoietic fated mesoderm markers (KDR+, KDR+PDGFRα−) at day 3 of differentiation, and FIG. 10(C) shows hematopoietic progenitor markers (CD34+CD43+, CD34+CD45+)/committed hematopoietic cells (CD34−CD43+, CD34−CD45+) at day 12 of differentiation. As concluded, cell line-to-cell line variation for cell differentiation may occur. Therefore, the higher number of iPSC clones generated from the MC based platform provides a higher chance to find the best clone for differentiation towards CD34+/CD43+/CD45+ hematopoietic progenitor cells for HSC transplantation therapy.

FIG. 11 shows the karyotyping of the representative reprogrammed MC-iPSCs from HFF-01, IMR90, PBMC, CD3+ T cells, and CD34+ cells by the ReprograMC approach.

FIG. 12, comprising FIGS. 12(A) and 12(B), shows the comparison of transduction efficiency and reprogramming efficiency for different cell sources with different reprogramming platforms (MNL and ReprograMC A & ReprograMC B), wherein FIG. 12(A) shows the transduction efficiency, and FIG. 12(B) shows the reprograming efficiency. **p<0.001; ***p<0.0001; ****p<0.00001. Error bars SEM, (n=3).

FIG. 13, comprising FIGS. 13(A), 13(B), 13(C), 13(D) and 13(E), shows the generation of HFF-01 derived MC-iPSCs with ReprograMC A, wherein FIG. 13(A) shows cell-covered MCs after 2-days culture under agitation (Scale bar=200 μm. Black arrows indicate cells attached on MCs), FIG. 13(B) shows a representative microscopic view of a well of a 6-well plate of cell-MC (Grey dots illustrated the Tra-1-60+ cell-MC. Scale bar=1 mm), FIG. 13(C) shows an individual Tra-1-60-stained cell-covered MC in TGP hydrogel at day 14 (Scale bar=50 μm. White arrow indicates Tra-1-60+ cell on a single MC.), FIG. 13(D) shows a representative image of cell-covered-MC aggregate expanded in 12-well ULA plate at day 28 (Scale bar=50 μm. White arrows indicate cell growth between MCs.), and FIG. 13(E) shows a representative image of cell-covered-MC aggregate expanded in 6-well ULA plate at day 35 (Scale bar=50 μm. White arrows indicate cell growth between MCs).

FIG. 14 shows the gene expression profiles during the initiation phase and maturation phase of reprogramming by quantitative PCR. Fold-changes relative to day 0 fibroblasts are depicted for monolayer and both ReprograMC A & ReprograMC B cultures. Expression levels that differ significantly at matching timepoints were depicted by horizontal brackets (ANOVA *p<0.01; **p<0.001; ***p<0.0001). Error bars SEM., (n=3).

FIG. 15 shows the gene expression profiles during the stabilization phase of the reprogramming process determined by quantitative PCR. Fold-changes relative to day 0 fibroblasts are depicted for monolayer and both ReprograMC A & ReprograMC B cultures. Expression levels that differ significantly at matching timepoints were depicted by horizontal brackets (ANOVA *p<0.01; **p<0.001; ***p<0.0001; ****p<0.00001). Error bars SEM, (n=3).

FIG. 16, comprising FIGS. 16(A), 16(B), 16(C), 16(D) and 16(E), shows the reprogramming of HFF-01 fibroblast by 3-factors only (Oct4, Sox2, and Klf4) on MNL and ReprograMC B approaches, wherein FIG. 16(A) shows comparison of reprogramming efficiency between MNL and ReprograMC, FIG. 16(B) is a flow cytometry analysis showing expression of pluripotent markers (Tra-1-60, Oct4, and mAb84) in the reprogrammed MC-iPSCs (3F-HFF01 to 3F-HFF12), FIG. 16(C) shows fold change of pluripotent and three germ-layer-specific genes compared with undifferentiated MNL-iPSCs, FIG. 16(D) shows staining of in vitro differentiated MC-iPSCs (3F-HFF01) for markers of mesoderm (SMA, α-smooth muscle actin), ectoderm (β-III tubulin) and endoderm (AFP, α-fetoprotein) (Scale bars: 200 μm), and FIG. 16(E) shows karyotyping of a representative clone (3F-HFF01). Normal 46 XY karyotypes by G-banding, 20 metaphase spreads were counted per sample.

FIG. 17, comprising FIGS. 17(A), 17(B), 17(C), 17(D) and 17(E), shows the induction of iPSCs from HFF-01 in monolayer cultures, wherein FIG. 17(A) shows representative brightfield images of generating iPSCs in monolayer cultures at different timepoints (days 14, 21, and 28), with the images showing a representative single colony was picked at day 14 (black circle) and plated on a well of LN-coated plate (Scale bars: 300 μm), FIG. 17(B) is a flow cytometry analysis showing expression of pluripotent markers (Tra-1-60, Oct4, and SSEA-4) in the reprogrammed MNL-iPSCs (MNL01 to MNL04), FIG. 17(C) shows fold change of pluripotent and three germ-layer-specific genes compared with undifferentiated MNL-iPSCs, FIG. 17(D) shows staining of in vitro differentiated MC-iPSCs (MNL01) for markers of mesoderm (SMA, α-smooth muscle actin), ectoderm (β-III tubulin) and endoderm (AFP, α-fetoprotein) (Scale bars: 200 μm), and FIG. 17(E) shows karyotyping of a representative clone (MNL01). Normal 46 XY karyotypes by G-banding, 20 metaphase spreads were counted per sample.

### DESCRIPTION OF EMBODIMENTS

The technology of generating induced pluripotent stem cells (iPSCs) holds great promise for revolutionizing regenerative medicine. The reprograming is conventionally done on monolayer cultures. However, this method results in low reprogramming efficiencies and require multistep handling which include enzymatically passaging to sub-culture the best clone for expansion and differentiation. In order to resolve these issues, the inventors have developed a microcarriers (MCs)-based reprogramming platform that offers automated, high-throughput, and large-scale production of induced pluripotent stem cells (iPSCs). The present disclosure also provides a technology for integration of reprogramming, expansion, and differentiation of iPSCs in a single platform.

A critical step for the entire process chain is the selection of high quality iPSCs clones in the culture dish. Frequently, selection is based on the morphology of the colonies analysed by phase contrast microscopy. The MCs platform being developed allow the sorting of the iPSCs colonies by size and are fully integrated into the automated production system.

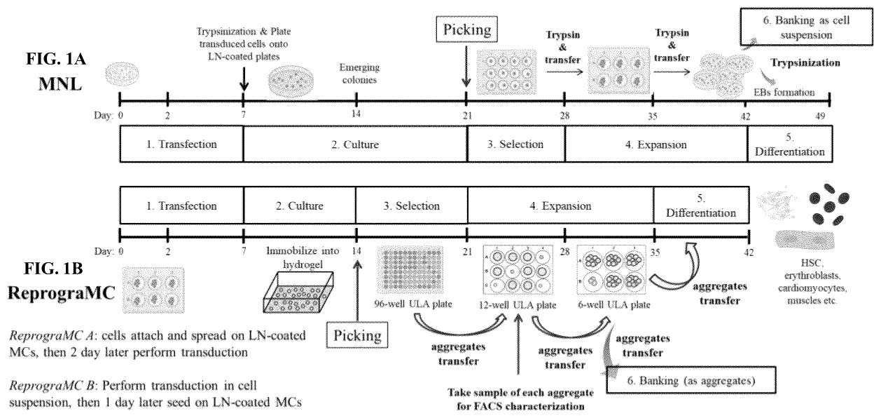

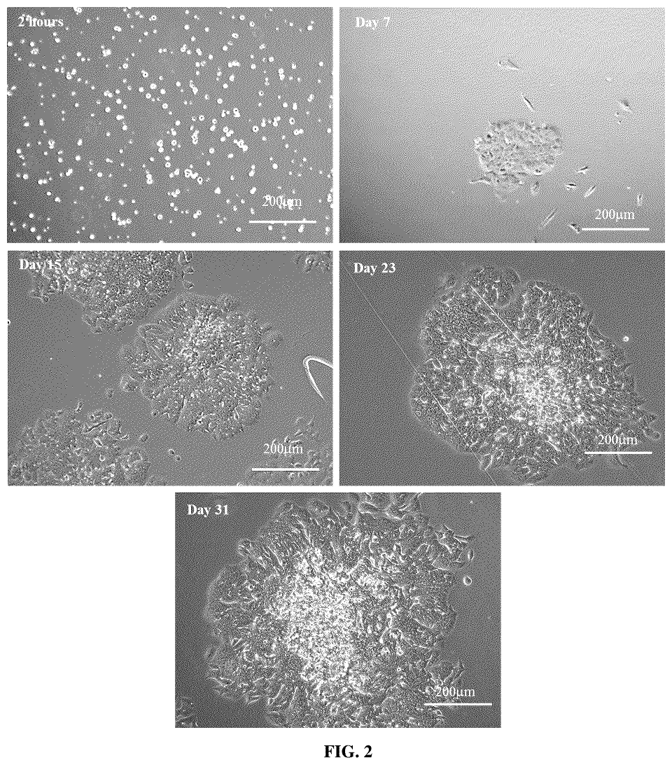

The present disclosure describes the development of a microcarriers (MCs) platform for somatic cell reprogramming to overcome the drawbacks from conventional monolayer reprogramming approach as aforementioned. FIG. 1(B) shows a flowchart of the MCs based iPSCs reprogramming platform (Methods A and B).

Two MCs platform approaches can be used: (i) ReprograMC B (FIG. 1(B), Method B): Transduction of the somatic cells with four transcription factors (Oct4, Sox2, Klf4 and c-Myc) or three transcription factors (Oct4, Sox2 and Klf4) followed by seeding the reprogrammed cells onto ECM-coated MCs, such as laminin-coated MCs. Other ECM such as vitronectin, fibronectin, heparan sulfate, and collagen, etc. can also be used; and (ii) ReprograMC A (FIG. 1(B), Method A): Seeding of the somatic cells on the ECM-MCs followed by transduction of the four transcription factors (Oct4, Sox2, Klf4 and c-Myc) or the three transcription factors (Oct4, Sox2 and Klf4). The MCs that can be used are, for example, positively-charged polystyrene microcarriers with a size of about 120 μm sourced from SoloHill® (see Examples for preparation of Solohill® PlasticPlus microcarriers). Other microcarriers such as alginate-based, dextran-based (DEAE and Cytodex™), collagen-based, gelatin-based, acrylamide-based, and glass-based as well as biodegradable MCs (such as poly-ε-caprolactone PCL and Poly(lactic acid-co-glycolic acid) PLGA), etc. can also be used. Due to the high volume-to-area ratio of the MCs, large numbers of iPSCs clones can be screened and selected.

Thus, in a first aspect, there is provided a method of reprogramming somatic cells into induced pluripotent stem cells (iPSCs), comprising:
(a) seeding the somatic cells on a plurality of microcarriers (MCs) to form cell-MC aggregates; and
(b) transducing transcription factors into the somatic cells of the cell-MC aggregates;
wherein step (a) and step (b) are carried out under continuous agitation.

Cell reprogramming is the process of reverting mature, specialized cells into undifferentiated cells, such as induced pluripotent stem cells (iPSCs). It can rejuvenate somatic cells by erasing the epigenetic memories and reconstructing a new pluripotent order. The basic phases of reprogramming include: (1) transduction of reprogramming transcription factors; (2) selection of undifferentiated cell or iPSC-like colonies; (3) expansion of the selected colonies; and (4) characterization of the expanded colonies. Therefore, the term “reprogramming” when used in relation to cellular reprogramming as described in the present disclosure refers to the process of converting a differentiated cell (such as a somatic cell) into an undifferentiated cell. The undifferentiated cell resulting from the reprogramming may be a pluripotent cell.

The term “seeding” when used in relation to a cell, refers to a process of contacting a cell with a material (for example, a microcarrier) or vessel such that the cell can undergo growth and expansion. In one example, the cell can attach to the material to undergo growth and expansion. The seeding process may be achieved, for example, by using inoculating loop, inoculating needle, pipette (such as a multichannel pipette, or an automated pipetting system), or any other methods known in the art. The source of the cell for seeding can be, but is not limited to, a frozen cell, a suspension cell, or an adherent cell. The suspension cell may be any cell known in the art that are able to float freely (suspended) in a culture medium during growth and/or non-growth phases. The suspension cell may also be contacted with a material (such as a microcarrier) for growth and/or expansion. The adherent cell may be any cell known in the art that undergoes growth and/or expansion when contacted with a material (such as a microcarrier) or a surface (such as the surface of a vessel). The adherent cell may be detached from the contacting material or surface and allowed to float or suspend in the culture medium before seeding. The source of the cell may be cultured as a single-cell suspension (pure cell culture having one type of cells) or mixed suspension (with more than one type of cells) before seeding. In one example, the source of the cell is a single-cell suspension. In one example, the single-cell suspension may be prepared using suspension cells. In another example, the single-cell suspension may be prepared using adherent cells that have been detached from the contacting material or surface that the cells had previously adhered to. In one example, the cells are contacted with a vessel. The vessel can be any vessel known in the art, such as, but is not limited to, petri dish, cell culture flask, cell culture tube, or multi-well cell culture plate. In one example, the multi-well cell culture plate can be a 96-well plate, 48-well plate, 24-well plate, 12-well plate, or 6-well plate. In one example, the multi-well cell culture plate is an ultra-low attachment (ULA)-coated plate. In one example, the cells are contacted with a carrier. In one example, the carrier is a microcarrier.

The term “somatic cell” refers to any cell of a living organism which is not a reproductive cell or germ line cell; the germ line cell being the cells in the sexual organs that produce sperm and eggs. The somatic cell can be any somatic cell which may be obtained using standard methods known in the art, from human or other mammals. For example, the somatic cell can be, but is not limited to, fibroblast, somatic stem cell, sertoli cell, endothelial cell, neuron, pancreatic islet cell, epithelial cell, hepatocyte, hair follicle cell, keratinocyte, hematopoietic cell, melanocyte, chondrocyte, lymphocyte, erythrocyte, macrophage, monocyte, mononuclear cell, muscle cell, and combinations thereof. Somatic stem cell refers to a cell that can give rise to a family of related cells only and/or may be reprogrammed into a pluripotent cell. The somatic stem cell may include, for example, mesenchymal stem cell, neural cell, hematopoietic stem cell and skin stem cell. In one example, the somatic cells are selected from the group consisting of human somatic cells, bovine somatic cells, and avian somatic cells. In one example, the somatic cells are selected from the group consisting of cells obtained from blood and/or bone marrow, cells obtained from skin biopsy, and fibroblasts. In one example, the somatic cells are cells obtained from blood and/or bone marrow. In another example, the somatic cells are cells obtained from skin biopsy. In another example, the somatic cells are fibroblasts. In one example, the somatic cells obtained from blood and/or bone marrow are selected from the group consisting of T cells, erythroblasts, peripheral blood mononuclear cells (PBMCs) and somatic stem cells (such as hematopoietic stem cells (HSCs)). In one example, the somatic cells are hematopoietic stem cells (HSCs). HSC is a progenitor cell that can develop into all types of blood cells, including white blood cells, red blood cells, and platelets. HSCs are found in the peripheral blood, umbilical cord blood, and the bone marrow. In one example, the HSC expresses CD34. In another example, the HSC expresses CD90. In another example, the HSC expresses CD43. In another example, the HSC express CD45. In another example, the HSC expresses any combination of CD90, CD43, CD45 and CD34. In another example, the HSC expresses CD90, CD43, CD45 and CD34. In another example, the somatic cells obtained from blood and/or bone marrow are HSCs. In another example, the somatic cells obtained from skin biopsy are selected from the group consisting of human foreskin fibroblasts (HFF), human dermal fibroblasts and human keratinocytes. In another example, the fibroblasts are human lung fibroblasts. In one example, the somatic cells are adherent somatic cells. In one example, the adherent somatic cells are adherent fibroblast cells. In another example, the adherent fibroblast cells are HFF-1 cells. In another example, the adherent fibroblast cells are IMR90 cells. In one example, the somatic cells are suspension somatic cells. In another example, the suspension somatic cells are PBMCs. In another example, the suspension somatic cells are CD3+ T cells. In another example, the suspension somatic cells are CD34+ cells.

The term “pluripotent cell” refers to cells that can give rise to all the cell types that make up the body. Exemplary pluripotent stem cells include embryonic stem cells and iPSC. In one example, the pluripotent cell is iPSC.

The term “microcarrier (MC)” refers to any particulate material capable of serving as a carrier for somatic cells to support attachment, growth and/or expansion of the cells. Regardless of its shape, MC possesses high surface area which allows for the attachment, growth, and/or expansion of the cells. In one example, the MC is in the form of a bead. In one example, the MC is in the form of a disk. The MC may be spherical or non-spherical. In one example, the MC is a spherical MC. The MC may be porous or non-porous. The MC may be uncharged or charged, and charged MCs may be positively charged or negatively charged. In one example, the MCs are selected from the group consisting of positively-charged polystyrene MCs, alginate-based MCs, dextran-based MCs, collagen-based MCs, gelatin-based MCs, acrylamide-based MCs, glass-based MCs, and biodegradable MCs. In one example, the biodegradable MCs are selected from the group consisting of poly-ε-caprolactone (PCL), Poly (lactic acid-co-glycolic acid) (PLGA) and Positively-charged Cytodex 1. In one example, the MCs are positively-charged polystyrene MCs. In one example, the MCs are coated by extracellular matrix (ECM). The coating of ECM enhances somatic cells attachment, growth and/or expansion. In one example, the ECM is selected from the group consisting of laminin, vitronectin, fibronectin, heparan sulfate, and collagen. In one example, the ECM is laminin. In one example, the ECM is Laminin521 (LN). In one example, the size of the MCs is 90-200 μm. In one example, the size of the MCs is 90-190 μm, or 90-180 μm, or 90-170 μm, or 90-160 μm, or 90-150 μm, or 90-140 μm, or 90-130 μm, or 90-120 μm, or 90-110 μm, or 90-100 μm, or 100-190 μm, or 100-180 μm, or 110-170 μm, or 120-160 μm, or 130-150 μm. In one example, the size of the MCs is about 90 μm, or about 100 μm, or about 110 μm, or about 120 μm, or about 130 μm, or about 140 μm, or about 150 μm, or about 160 μm, or about 170 μm, or about 180 μm, or about 190 μm, or about 200 μm. In one example, the size of the MCs is about 120 μm. In one example, the MCs used may comprise of a single size. In another example, MCs of different sizes may also be used at the same time. For example, the MCs may comprise a mixture of two or more sizes selected from about 90 μm, about 100 μm, about 110 μm, about 120 μm, about 130 μm, about 140 μm, about 150 μm, about 160 μm, about 170 μm, about 180 μm, about 190 μm, and about 200 μm.

The term “transduction” or “transfection” refers to the introduction of foreign material, such as genetic material or transcription factors, into a cell. The transduction may be achieved via any methods known in the art and using any agents known in the art. In one example, the transduction is done via an agent selected from the group consisting of virus, protein, plasmids, PiggyBac, and small molecules. In one example, the protein is an Embryonic Stem Cell (ESC)-derived extract protein, or a Cell-Penetrating Peptide selected from the group consisting of a designed peptide, a natural protein-derived peptide, and a chimeric peptide. In one example, the plasmid is an expression plasmid comprising the complementary DNAs (cDNAs) of Oct3/4, Sox2, and Klf4. In one example, the small molecule is selected from the group consisting of Ascorbic acid, Valproic acid, and Sodium butyrate. In one example, the transduction is done via a virus. In one example, the virus is selected from a group consisting of respirovirus, lentivirus, retrovirus, and adenovirus. In one example, the respirovirus is Sendai virus.

In one example, the transduction is achieved using one or more transcription factors capable of affecting the activity or regulation of genes, for example, by increasing or reducing the expression of the genes that encode pluripotent markers. The transcription factors are involved, for example, in the process of converting or transcribing nucleic acids, such as from DNA into RNA. In one example, the transcription factors are proteins. The transcription factors may be selected from the group consisting of Oct3/4, Sox2, Klf4, Nanog, c-Myc, and LIN28. In one example, the transcription factors comprise Oct4, Sox2, Klf4, and c-Myc. In one example, the transcription factors comprise Oct4, Sox2 and Klf4. In one example, the transcription factors consist of Oct4, Sox2 and Klf4. In one example, the transcription factors exclude c-Myc. In one example, the transcription factors comprise Oct4, Sox2 and Klf4 and exclude c-Myc. In one example, the transcription factors further comprise one or more of Nanog, c-Myc, and LIN28. In one example, the transcription factors further comprise one or more of Nanog and LIN28.

Agitation is important in transduction on MCs. Agitation is applied throughout the whole transduction process, before and after the transduction. The mixing of the microcarriers by agitation allows the transducing agent to enter the cells more effectively to reprogram them. In one example, the transducing agent is a virus. In one example, the mixing of the microcarriers by agitation allows the viruses to get into the cells more effectively to reprogram them. In one example, the reprogrammed cells are firmly attached onto the laminin coated beads which stabilize their growth. In one example, the agitation is a continuous agitation. The agitation may be achieved by using any method, machine or apparatus that is known in the art. The agitation may be performed at a speed that allows optimum mixing of the microcarriers. In one example, the agitation is performed at 50-125 revolutions per minute (rpm). In one example, the agitation is performed at 50-75 rpm, or 50-80 rpm, or 50-90 rpm, 50-100 rpm, or 50-110 rpm, or 50-125 rpm, or 60-75 rpm, or 60-80 rpm, or 60-90 rpm, or 60-100 rpm, or 60-110 rpm, or 60-125 rpm, or 75-90 rpm, or 75-100 rpm, or 75-110 rpm, or 75-125 rpm, or 80-90 rpm, or 80-100 rpm, or 80-110 rpm, or 80-125 rpm, or 90-100 rpm, or 90-110 rpm, or 90-125 rpm, or 100-110 rpm, or 110-125 rpm. In one example, the agitation is performed at 75-110 rpm. In one example, the agitation is performed at 100-110 rpm. In one example, the agitation is performed at about 50 rpm, or about 60 rpm, or about 75 rpm, or about 80 rpm, or about 90 rpm, or about 100 rpm, or about 110 rpm, or about 125 rpm. In one example, the agitation is performed at about 75 rpm.

In one example, the agitation occurs in a stirred bioreactor system. In contrast to monolayer approach, MCs, which has increased cell surface area, leads to an increased yield of cells which has the ability to grow in bioreactor conditions at any stage. This means that the whole reprogramming process, from transduction to iPSC expansion, has the potential to be performed in stirred bioreactor systems, with controlled dissolved oxygen, temperature, and pH. A controllable culture environment enables control of the reproducibility and repeatability of the reprogramming run. In one example, the stirred bioreactor system comprises controlled conditions of dissolved oxygen, temperature, and pH. For example, hypoxia is known to enhance the efficiency of reprogramming; therefore performing the reprogramming process in stirred bioreactor systems enables control of the dissolved oxygen to a level (such as 5% oxygen) to improve efficiency of reprogramming.

The method of the first aspect described herein may further comprise:
(c) immobilizing the cell-MC aggregates into a hydrogel;
(d) selecting the cell-MC aggregates showing fast cell growth or expressing pluripotency markers;
(e) expanding the selected cell-MC aggregates by adding fresh MCs to the selected cell-MC aggregates; and
(f) characterising the cell-MC aggregates to determine whether the somatic cells on the MCs have been reprogrammed to iPSCs.

The progression of the reprogramming can be monitored by immobilizing the cell-MC aggregates as described in step (c) of the method disclosed herein. The term “immobilize” in the present disclosure refers to fixing the cell or cell-MC aggregates onto a carrier to restrict its mobility. The process of immobilization can be achieved with any methods known in the art. Examples of immobilization methods may include, but are not limited to, the use of materials such as hydrogels, which may include agarose, temperature-sensitive hydrogel and other types of hydrogels known in the art. The agarose may have high melting point or low melting point. In one example, the agarose is a low melting point agarose. In one example, the hydrogel used for immobilizing the cell-MC aggregate is an agarose gel. In one example, the hydrogel used for immobilizing the cell-MC aggregate is a 0.5% (w/v) agarose gel. In one example, the hydrogel used for immobilizing the cell-MC aggregate is a 0.5% (w/v) low melting point agarose gel. The temperature-sensitive hydrogel may be, for example, a thermoreversible hydrogel (such as thermoreversible gelation polymer (TGP)). In one example, the hydrogel used for immobilizing the cell-MC aggregate is a temperature-sensitive hydrogel. In one example, the hydrogel used for immobilizing the cell-MC aggregate is a thermoreversible hydrogel. In one example, the hydrogel used for immobilizing the cell-MC aggregate is a TGP. Immobilization of the cell-MC aggregates into hydrogel (such as 0.5% (w/v) agarose gel) enables easy monitoring the progression of cell growth. Moreover, in situ staining for the expression of pluripotent markers, such as Tra-1-60, in hydrogel culture allows easy identifying, scoring, selecting, and picking of iPSCs-like cell-MC aggregates. After immobilization, the cell-MC aggregates can be visualized using any known method known in the art. In one example, the plates containing the cell-MC aggregates can be simply visualized by microscopic observation (real-time video recording can also be used) to monitor the progression of the reprogramming.

After immobilization, cell-MC aggregates that show fast cell growth or express pluripotency markers are selected as described in step (d) of the method disclosed herein.

The term “fast cell growth” in the method described herein means that the size of cell-MC aggregate increases faster than other cell-MC aggregates in the same sample within a certain period, usually within 7 days. In one example, the size of cell-MC aggregate increases faster within 1 day, or 2 days, or 3 days, or 4 days, or 5 days, or 6 days, or 7 days. In one example, the size of cell-MC aggregate increases faster within 7 days. The increase in size of the fast-growing cell-MC aggregate compared to the size of the initial cell-MC aggregate is at least 2 times, or at least 3 times, or at least 4 times, or at least 5 times, or at least 6 times, or at least 7 times, or at least 8 times, or at least 9 times, or at least 10 times. In one example, the increase in size of the fast-growing cell-MC aggregate compared to the size of the initial cell-MC aggregate is at least 2 times. In one example, the increase in size of the fast-growing cell MC aggregate is at least 2 times and occurs within 7 days. In one example, there is no upper limit on the increase in size of the fast-growing cell-MC aggregate compared to the size of the initial cell-MC aggregate.

Pluripotent markers are molecular markers, such as mRNA and cell surface antigen or protein, that may be used to determine the pluripotent status of cells. The pluripotency markers can be, but is not limited to Tra-1-60, Oct4, SSEA-4, Tra-1-81, and mAb84. In one example, the pluripotency marker is selected from the group consisting of Tra-1-60 and Tra-1-81. In one example, the pluripotent marker is Tra-1-60. In one example, the pluripotency markers are Tra-1-60, Oct4 and SSEA-4.

The selected cell-MC aggregates that show fast cell growth or express the pluripotent markers are then expanded by adding fresh MCs as described in step (e) of the method disclosed herein. The term “expansion” refers to a process of increasing the number of cells or increasing the cell population. The process of expansion can be achieved by any method known in the art. For example, the process of expansion can be achieved by subculturing. In one example, the expansion of the selected cell-MC aggregates is achieved by sub-culturing. In one example, the sub-culturing involves the transferring of the cell-MC aggregates from the 96-well plate to a 12-well plate and then to a 6-well plate. In one example, subsequent expansion of the picked Tra-1-60⁺ iPSC-MC can be straightforwardly carried out by sub-culturing the Tra-1-60⁺ iPSC-MC aggregates from 96-well plate to 12-well plate, then 6-well plate. In one example, no enzymatic dissociation procedure is involved for sub-culturing and monitoring. In one example, fresh MCs are added from each cell-MC aggregate sub-culturing process.

The term “fresh MCs” refers to new MCs which have not been used in any way. In one example, the term “fresh MCs” refers to new MCs coated with ECM. In one example, the term “fresh MCs” refers to new MCs coated with ECM selected from the group consisting of laminin, vitronectin, fibronectin, heparan sulfate and collagen. In one example, the term “fresh MCs” refers to new laminin-coated MCs. In one example, the term “fresh MCs” refers to new Laminin521 (LN)-coated MCs.

The expanded cell-MC aggregates are then characterized to determine whether the somatic cells on the MCs have been reprogrammed to iPSCs as described in step (f) of the method disclosed herein. The characterization of the cell-MC aggregates can be, but is not limited to, morphological, expression of pluripotent genes or markers, FACS analysis of pluripotency, evaluation of cell growth capacity, karyotyping, in vitro tri-lineage differentiation and any other types of cell characterization known in the art. In one example, the characterization of the cell-MC aggregates to determine whether the somatic cells on the MCs have been reprogrammed to iPSCs is selected from the group consisting of FACS analysis for pluripotency, evaluation of cell growth capacity, evaluation of multi-passage stability, karyotyping, and evaluation of in vitro tri-lineage differentiation. In one example, the karyotypic is performed using a G-banding assay. In one example, characterization of the cell-MC aggregates can be done simply by sampling some of the aggregates from the well, no trypsinization is needed.

All the procedures can be integrated with an automated machine for a more high-throughput production, expansion, and differentiation system of iPSCs. In one example, the steps of the method described herein are integrated with an automated machine. The automated machine may include, but is not limited to, ClonePix™ from Molecular Devices and ALS CellCelector™ platform. In one example, the automated machine is ClonePix™ System from Molecular Devices. In one example, the automated machine is ClonePix™ FL from Molecular Devices.

In a second aspect, there is provided a method of producing, selecting, expanding, characterizing, and differentiating iPSCs, comprising:
carrying out steps (a)-(b) of the first aspect;
(c) immobilizing the cell-MC aggregates into a hydrogel;
(d) selecting the cell-MC aggregates showing fast cell growth or expressing pluripotency markers;
(e) expanding the selected cell-MC aggregates by adding fresh MCs to the selected cell-MC aggregates;
(f) characterising the cell-MC aggregates to determine whether the somatic cells on the MCs have been reprogrammed to iPSCs; and
(g) differentiating the cells of the cell-MC aggregates toward functional cells.

The selected, expanded and characterized cells of the cell-MC aggregates may be further differentiated towards functional cells as described in step (g) of the method disclosed herein. The differentiation may be achieved by any methods or protocols known in the art. The term “functional cells” refers to any type of differentiated cells in the body. For example, the differentiated cells can be, but not limited to, cardiomyocytes, neural progenitor cells, neurons, erythroblasts, HSC cells, retinal pigment epithelium, photoreceptors, beta-islet, T cells and NK cells. In one example, the differentiated cells are selected from the group consisting of cardiomyocytes, erythroblasts and HSCs. In one example, the cardiomyocytes are cTnT+ cells. In one example, the erythroblasts are DRAQ5+ cells. In one example, the HSC cells are CD34+/CD43+ cells or CD32+/CD45+ cells. In one example, the differentiation of cells of the cell-MC aggregates towards functional cells is via the formation of Embryoid bodies (EBs)-like cell-MC aggregates. In one example, the formation of EBs-like cell-MC aggregates is facilitated by continuous agitation. In one example, the formation of EBs-like cell-MC aggregates is facilitated by centrifugation. The term “Embryoid bodies (EB)” refers to aggregates of pluripotent cells that are induced to differentiate. Therefore, “EBs-like” aggregates refer to aggregates of pluripotent cells. However, they are not being induced for differentiation, but still maintain their pluripotency. In one example, the Embryoid Bodies (EBs)-likes cell-MC aggregates can be directly subjected to differentiation without any trypsinization. In one example, the differentiation of the EBs-likes cell-MC aggregates is done by simply changing the iPSCs growth medium to appropriate differentiation medium.

In a third aspect, there is provided a method of reprogramming somatic cells selected from the group consisting of fibroblasts IMR90, fibroblasts HFF-01, PBMC, CD3+ T cells and CD34+ hematopoietic stem cells (HSCs) into induced pluripotent stem cells (iPSCs), comprising:
(a) seeding the somatic cells on a plurality of microcarriers (MCs) to form cell-MC aggregates;
(b) transducing transcription factors into the somatic cells of the cell-MC aggregates using Sendai virus;
(c) immobilizing the cell-MC aggregates into a hydrogel;
(d) selecting the cell-MC aggregates showing fast cell growth or expressing pluripotency markers;
(e) expanding the selected cell-MC aggregates by adding fresh MCs to the selected cell-MC aggregates; and
(f) characterising the cell-MC aggregates to determine whether the somatic cells on the MCs have been reprogrammed to iPSCs;
   wherein step (a) and step (b) are carried out under continuous agitation; and
   wherein the transcription factors comprise Oct4, Sox2, c-Myc, and Klf4.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXAMPLES

Non-limiting examples of the disclosure will be further described in greater detail by reference to specific Examples, which should not be construed as in any way limiting the scope of the disclosure.

### Materials and Methods

### Cell Culture

Human fibroblast lines HFF-01 (ATCC® SCRC-1041™) and IMR-90 (ATCC®CCL-186™) were propagated using α-MEM media containing 10% Fetal Bovine Serum (FBS) and 1% Penicillin-Streptomycin (PS) (designated as α10) in a 37° C. 5% CO₂ humidified incubator. Passage 2-5 was used for reprogramming. Single cell suspension was generated by using 0.05% trypsin/0.025% EDTA (ThermoFisher Scientific).

Human frozen PBMCs were purchased from ATCC (PCS-800-011™) and were cultured in PBMC medium consisting of StemPro®-34 serum-free medium supplemented with stem cell factor (SCF, 100 ng/mL), flt-3 Ligand (Flt-3L; 100 ng/ml), interleukin (IL)-3 (20 ng/ml), and IL-6 (10 ng/ml; all from Peprotech) for 4 days at 37° C. in 5% CO₂ incubator for cell recovery. CD3+ T-cells were isolated from the cultured PBMCs by using EasySep Human T-cells isolation kit (StemCell Technologies), according to the manufacturer's instruction. Isolated CD3+ T-cells were cultured in X-VIVO™ 10 medium (Lonza) supplemented with IL-2 (50IU per 106 cells; Peprotech) for 4 days at 37° C. in 5% CO₂ incubator. CD34+ cells from PBMC were isolated using CD34 MicroBead Kit (Miltenyi Biotech) according to the manufacturer's protocol. Isolated CD34+ cells were expanded in StemSpanII Serum-Free Expansion Medium (SFEM) (StemCell Technologies), supplemented with Flt3, SCF, TPO (300 ng/ml each), IL-6 (100 ng/ml) and IL-3 (10 ng/ml) (all from Peprotech), for 4 days at 37° C. in 5% CO₂ incubator.

### Materials

CytoTune®-iPS 2.0 Sendai virus Reprogramming kit was purchased from ThermoFisher Scientific. The Sendai virus encodes the four transcription factors: Oct4, Sox2, Klf4 (KOS) and c-Myc. The calculated volumes of each of the three SeV vectors (KOS, c-Myc, and Klf4) were added to the cells using a Multiplicity of Infection (MOI) of 5:5:3, respectively.

Solohill® Plastic Plus (PP+) microcarriers was purchased from Sartorius. Laminin521 (LN) was bought from Biolamina. Chemically synthesized thermoreversible hydrogel (TGP) was obtained from Mebiol Inc.

### Preparation of LN521-Coated MCs and Plates

Prior to the start of the experiment, Solohill® PP+ MCs were prepared according to the manufacturer's instructions, including sterilization. MCs were coated with Laminin521 (LN; Biolamina) as previously described (Lam, Li et al. 2015). Briefly, MC coating was prepared by adding 20 μg pf LN521 in 22.5 mg of Solohill® PP+ in PBS at 4° C. overnight under rotation. The coated MCs was then designated as LN-coated MCs. Plates were coated at 0.5 μg LN521 per cm² according to manufacturer's instructions.

### Sendai Virus (SeV) Reprogramming

FIG. 1 Visual representation of reprogramming methods

### Reprogramming by Conventional Method in Monolayer Cultures (MNL)

Adherent HFF-1 and IMR90 fibroblasts: Transduction was done by adding 4 μg polybrene (Sigma-Aldrich) and SeV vectors encoding the 4 transcription factors (Oct4, Sox2, Klf4, and c-Myc) or 3 transcription factors (Oct4, Sox2, and Klf4) to 3×10⁵ cells and maintained in a well of a 6-well tissue culture plate containing 1 ml of α10 medium. Following overnight incubation (Day 1 post-transduction), the spent medium (with SeV) was replaced with fresh α10 medium every other day for a week. Seven days after transduction (Day 7), the cells were trypsinized and plated onto a separate well in a 6-well tissue LN-coated plate. On the next day, spent medium was replaced with Essential 8™ medium (E8; ThermoFisher Scientific). Medium was changed daily. Colonies with an iPSC-like appearance were thereafter manually isolated based on morphology and cultured as iPSCs on LN-coated plate with 5 ml of mTeSR™ 1 medium (mT; StemCell Technologies).

Suspension PBMC, CD3+ T-cells and CD34+ cells: Suspended 5×10⁵ PBMC, CD3+ T-cells or CD34+ cells were plated per well of a 24-well ULA plate in 300 μl of their corresponding cell growth medium. Subsequently, the SeV vectors encoding 4 transcription factors were added and the plates were placed in a CO₂ incubator overnight. Following the overnight incubation (Day 1 post-transduction), the spent medium (with SeV) was removed by centrifugation and replaced with fresh corresponding cell growth medium. Plates were placed in a CO₂ incubator for 2 days. Thereafter, the cultures were treated as described above for adherent cells.

### Reprogramming by Novel ReprograMC Approach in Microcarrier Cultures (MC): ReprograMC A and ReprograMC B (FIG. 1(B))

ReprograMC A (Method A, FIG. 1(B))—Direct reprogramming of cells on MCs: 3×10⁵ single-cell suspensions of HFF-1 and IMR90 were added in a 6-well ULA plate containing 20 mg of LN-coated MCs and 5 ml of α10 medium. The plate was agitated (100-110 rpm) in a 37° C., 5% CO₂ shaker incubator (New Brunswick™ S41i Incubator Shaker) under agitation. Two days after, when cell-covered-MC (cell-MC) were obtained, the medium was changed to fresh α10 medium containing the SeV vectors encoding KOS, c-Myc, and Klf4. Plate was placed in the CO₂ shaker incubator under agitation (100-110 rpm). Following overnight incubation under agitation (Day 1 post-transduction), the SeV-containing medium was removed by simply allowing the cell-MC to settle down and replacing with fresh E8 medium. The cell-MC were cultured in the CO₂ shaker incubator under agitation (100-110 rpm) with fresh E8 medium changed every other day for a week. Seven days after transduction (Day 7 post-transduction), the cell-MC were collected, resuspended in 1 ml of mT medium, and subsequently mixed with TGP hydrogel on ice. Immediately, the cell-hydrogel mixture was transferred evenly into 6 wells of a 6-well tissue culture plate. Since the hydrogel is temperature-sensitive, all procedures were done on ice. Thereafter, the plate was put in room temperature for 10 min to allow the TGP hydrogel to solidify. mT medium was then overlaid on the TGP hydrogel and the plate was incubated in a 37° C., 5% CO₂ incubator for 7 days with daily mT medium changes.

ReprograMC B (Method B, FIG. 1(B))—Single-cell suspension reprogramming followed by MC seeding: Single-cell suspensions of 3×10⁵ HFF-1 and IMR90 or 5×10⁵ cells of PBMC, CD3+ T-cells and CD34+ cells were plated per well of a 6-well ULA plate with 2 ml of the corresponding cell growth medium (without MCs). Subsequently, the cell suspension was transduced with SeV vectors encoding KOS, c-Myc, and Klf4 and placed in the CO₂ shaker incubator under agitation (100-110 rpm) (Day 0). After 24 hours (Day 1 post-transduction), the SeV-contained spent medium was removed by centrifugation and the culture was replenished with fresh corresponding cell growth medium (2 ml) and transferred to a well of 6-well ULA containing 20 mg of LN-coated MCs and 5 ml E8 medium. Afterwards, the culture was treated as described in Method A above.

### Live-Cell Immunofluorescence Staining of Tra-1-60 Positive Cells on MC in Hydrogel

Live-staining with StainAlive Tra-1-60 (DyLight™488; Stemgent) was used to identify the onset of Tra-1-60 expression, a marker associated with pluripotency. Briefly, fresh mT media containing 5 μg of StainAlive Tra-1-60 antibody was added to the hydrogel culture and incubated for 30 minutes in a 37° C. 5% CO₂ incubator. After two washes with fresh warm mT medium, the Tra-1-60-stained cells in the hydrogel were analyzed using ClonePix™ System (Molecular Devices).

### Selection and Expansion of Tra-1-60 Positive Cell-MC

Following live stains with StainAlive Tra-1-60 antibody, the positively stained cell-MC (green color) were identified and marked for picking using ClonePix™ System. The marked cell-MC were picked accordingly and transferred into a separate well of a 96-well ULA plate (with 200 μl mT and 0.5 mg LN-coated MCs) under a stereomicroscope using 200 μl pipette tips (tips were changed after each pick to avoid cross-contamination of cells). The 96-well ULA plate was then incubated in a 37° C. 5% CO₂ incubator for 7 days under static conditions. On day 7, live-staining with StainAlive Tra-1-60 was performed again in the 96-well ULA plate to identify the growing pluripotent cell on MCs (in aggregates) under a fluorescence microscope. The growing and Tra-1-60+ cell-MC aggregates were selected (size increase at least 2 times that of the initial aggregate) and transferred into a separate well of a 12-well ULA plate (with 3 ml mT and 8 mg LN-coated MCs), using 1 ml pipette tips to avoid breaking down the cell-MC aggregates. The 12-well ULA plate was then incubated in a 37° C. 5% CO₂ incubator for another 7 days under static conditions. After 7-days incubation, fast-growing cell-MC aggregates (size increase at least 2 times of initial aggregate plated in the 12-well ULA plate) were selected and transferred into a separate well of 6-well ULA plate (with 5 ml mT and 20 mg LN-coated MCs), using 1 ml pipette tips. The cell aggregates should break down into smaller aggregates gently by the 1 ml pipette tips. The 6-well ULA plate with cell-MC aggregates was then incubated in a 37° C. 5% CO₂ incubator for another 7 days under agitation (100-110 rpm). The expanded cell-MC aggregates (MC-iPSCs) were then harvested for characterization.

### Transduction and Reprogramming Efficiency

Transduction efficiency was evaluated by the expression of enhanced green fluorescent protein (GFP) using Hoechst 33342 (ThermoFisher Scientific) and propidium iodide (PI; ThermoFisher Scientific) by an image cytometry, NucleoCounter® NC-3000 (ChemoMetec) according to the manufacturer's instruction. Briefly, cells were transduced with CytoTune®-EmGFP Sendai Fluorescence Reporter (ThermoFisher Scientific) by MNL and ReprograMC approaches as above mentioned. After 24 hours of transduction, cells were collected and stained with 10 μg/ml Hoechst 33342 for 15 minutes. PI (10 μg/ml) was then added prior to loading into the Nucleocounter. Images acquired from the NucleoCounter were then quantified by the NucleoView™ software. In the software, cells were located using Hoechst 33342 and non-viable cells were stained with PI. The percentage of GFP expressing live cells was then calculated by the software.

Reprogramming efficiency was calculated as the number of emerging Tra-1-60+ iPSCs colonies (MNL method) or aggregates (MC methods) at Day 14 per starting cell number (Day 0).

$$Reprogramming efficiency = \frac{No . o f Tra - 1 - 60 + iPSCs at Day 14}{No . of input starting cells at Day 0} \times 100 \%$$

### Time Course Gene Expression Analysis

Gene expression by real-time polymerase chain reaction (RT-qPCR) was measured at multiple time points during cellular reprogramming. A set of known genes related to cellular reprogramming were examined, which included Thy1, Snail1, Snail2, CD44, Alp, β-catenin, Nanog, Lin28A, Sall4, E-cadherin, EpCAM, Oct3/4, Sox2, Dppa4, Klf4, GADPH, Nodal, GDF3 and DNMT3B. Briefly, RNA was extracted from cells at different time points after transduction (days 1, 2, 3, 4, 7, 14, 21, and 28) using an RNA extraction kit (RNeasy Mini Kit; Qiagen) in accordance with the manufacturer's instructions. The RNA was reverse transcribed into cDNA using Superscript II Reverse Transcriptase (ThermoFisher Scientific). The cDNA was mixed with Power SYBR Green PCR Master Mix (ThermoFisher Scientific) and 200 nM of the specific primers of the genes listed in Table 1. The reaction was carried out on an Applied Biosystems™ QuantStudio™ 3 Real-Time PCR System using the following cycling conditions: 50° C. for 2 minutes, 95° C. for 10 minutes, following by 40 cycles of 95° C. for 15 second and 60° C. for 1 minute. Fold change of each gene was referenced against the same gene prior to reprogramming (day 0).

TABLE 1
List of primer sets of genes used, which are commonly expressed during the
phases of reprogramming.

| SEQ ID NO | Sequence Name | Primer Sequence (5′-3′) |
|---|---|---|
| 1 | Thy1 Forward Primer | GAAGGTCCTCTACTTATCCGCC |
| 2 | Thy1 Reverse Primer | TGATGCCCTCACACTTGACCAG |
| 3 | Snail1 Forward Primer | TGCCCTCAAGATGCACATCCGA |
| 4 | Snail1 Reverse Primer | GGGACAGGAGAAGGGCTTCTC |
| 5 | Snail2 Forward Primer | ATCTGCGGCAAGGCGTTTTCCA |
| 6 | Snail2 Reverse Primer | GAGCCCTCAGATTTGACCTGTC |
| 7 | CD44 Forward Primer | CCAGAAGGAACAGTGGTTTGGC |
| 8 | CD44 Reverse Primer | ACTGTCCTCTGGGCTTGGTGTT |
| 9 | Alp Forward Primer | CCTGATGGAGATGACAGAGGCT |
| 10 | Alp Reverse Primer | TCAGTGAGTGCCTGGTAAGCCA |
| 11 | beta-catenin Forward Primer | CACAAGCAGAGTGCTGAAGGTG |
| 12 | beta-catenin Reverse Primer | GATTCCTGAGAGTCCAAAGACAG |
| 13 | Nanog Forward Primer | CTCCAACATCCTGAACCTCAGC |
| 14 | Nanog Reverse Primer | CGTCACACCATTGCTATTCTTCG |
| 15 | Lin28A Forward Primer | CCAGTGGATGTCTTTGTGCACC |
| 16 | Lin28A Reverse Primer | GTGACACGGATGGATTCCAGAC |
| 17 | Sall4 Forward Primer | GAAACCACATCCTTCCAGGCAC |
| 18 | Sall4 Reverse Primer | GATAAACGTGGAAGGGAGACTG |
| 19 | E-cadherin Forward Primer | CCTCCTGAAAAGAGAGTGGAAG |
| 20 | E-cadherin Reverse Primer | TGGCAGTGTCTCTCCAAATCCG |
| 21 | EpCAM Forward Primer | GCCAGTGTACTTCAGTTGGTGC |
| 22 | EpCAM Reverse Primer | CCCTTCAGGTTTTGCTCTTCTCC |
| 23 | OCT3/4 Forward Primer | CCTGAAGCAGAAGAGGATCACC |
| 24 | OCT3/4 Reverse Primer | AAAGCGGCAGATGGTCGTTTGG |
| 25 | SOX2 Forward Primer | GCTACAGCATGATGCAGGACCA |
| 26 | SOX2 Reverse Primer | TCTGCGAGCTGGTCATGGAGTT |
| 27 | Dppa4 Forward Primer | CTCCACAGAGAAGTCGAGGGAA |
| 28 | Dppa4 Reverse Primer | GGTTGTCAGTGTGCTCTGCCTT |
| 29 | Klf4 Forward Primer | CATCTCAAGGCACACCTGCGAA |
| 30 | Klf4 Reverse Primer | TCGGTCGCATTTTTGGCACTGG |
| 31 | GAPDH Forward Primer | GTCTCCTCTGACTTCAACAGCG |
| 32 | GAPDH Reverse Primer | ACCACCCTGTTGCTGTAGCCAA |
| 33 | Nodal Forward Primer | TGGCGTACATGCTGAGCCTCTA |
| 34 | Nodal Reverse Primer | CCTCTTGTTGGCTCAGGAAGGA |
| 35 | GDF3 Forward Primer | GTCTCCCGAGACTTATGCTACG |
| 36 | GDF3 Reverse Primer | AGTAGAGGAGCTTCTGCAGGCA |
| 37 | DNMT3B Forward Primer | TAACAACGGCAAAGACCGAGGG |
| 38 | DNMT3B Reverse Primer | TCCTGCCACAAGACAAACAGCC |

### Characterization of Reprogrammed MC-iPSCs

### Flow Cytometry

Flow cytometry analysis was performed with the extracellular antigen Tra-1-60 (Millipore), intracellular transcription factor Oct4 (R&D Systems) and stage-specific embryonic antigen-4 (SSEA-4; ThermoFisher Scientific). Briefly, MC-iPSCs cells were first trypsinized from MCs with TrypLE™ Express to form a single-cell suspension and then filtered through a 40-μm sieve (BD Biosciences) to remove cell debris and microcarriers. For MNL-iPSCs, a single-cell suspension was obtained from monolayer culture using TrypLE™ Express. Thereafter, all samples were fixed and permeabilized with a Fix and Perm Cell Permeabilization reagent kit (ThermoFisher Scientific) according to the manufacturer's instructions. During the 15-minute permeabilization step, mouse primary antibodies Tra-1-60 (1:50), Oct-4 (1:20), and SSEA-4 (1:100) were incubated together with the kit's Reagent B. Cells were subsequently washed with 1% BSA/PBS, followed by 15-minute incubation in the dark with a 1:500 dilution of FITC-conjugated goat anti-mouse antibody (DAKO). Finally, cells were washed and resuspended in 1% BSA/PBS for analysis on a NovoCyte Flow Cytometer (ACEA biosciences). Results were analyzed with FlowJo software (Tree Star), with gating selected at the point of intersection between the marker and isotype control.

### Spontaneous in Vitro Differentiation

Spontaneous in vitro differentiation and embryonic bodies (EBs) formation was carried out to determine whether MC-iPSC cells cultured on MCs retain their ability to differentiate into the three germ layers. Briefly, MC-iPSC aggregates were cultured as EBs for 7 days in differentiation medium [Knockout™ DMEM (Gibco) with 15% FBS (Gibco)] on non-adherent dishes and subsequently re-plated on 0.1% gelatinized plates for another 14 days.

Immunostaining was carried out to identify the three germ layers, with α-smooth muscle actin, SMA (Sigma), β-III tubulin (Millipore), and α-fetoprotein, AFP (Sigma), as previously described (Lam, Li et al. 2015). Briefly, the differentiated cells were fixed with 4% paraformaldehyde for 15 minutes and blocked for 2 hours in PBS containing 0.1% Triton X-100, 10% goat serum, and 1% BSA. Cells were then probed with primary antibodies SMA (1:400), β-III tubulin (1:1000), and AFP (1:250) for 1 hour and secondary FITC-conjugated antibody for another 2 hours at room temperature in dark. A fluorescent mounting medium with DAPI (Vectashield) was added to cover the cells. Following 1-hour incubation the cells were visualized with Axiovert 200M fluorescence microscope (Carl Zeiss).

The expression of three germ layers markers is analyzed by RT-qPCR. Briefly, RNA was extracted from undifferentiated and differentiated MC-iPSCs. The RNA was then reverse transcribed into cDNA and subsequently mixed with Power SYBR Green PCR Master Mix and 200 nM of the specific primers of the following genes, OCT-4, AFP, GATA6, Hand1, Nkx2.5, PAX6, SOX1, and GAPDH (housekeeping gene), as previously described (Chen, Chen et al. 2011). The PCR reaction was carried out using the following cycling conditions: 50° C. for 2 minutes, 95° C. for 10 minutes, following by 40 cycles of 95° C. for 15 second and 60° C. for 1 minute. Fold change of each gene was referenced against the same gene prior to differentiation of MC-iPSCs.

### Karyotype Analysis

To assess chromosomal stability of the MC-iPSCs clones, karyotyping of all clones by G-banding assay at passage 10 (using bromodeoxyuridine/colcemid) was performed by the cytogenetics lab in Singapore General Hospital.

### Cardiac Differentiation

Cardiac differentiation using a Wnt differentiation protocol was previously described (Lam, Chen et al. 2014). Briefly, aggregates (used as EBs) of 1×10⁶ MC-iPSCs on MCs were cultivated in RPMI+B27 medium without insulin (IN−) supplemented with 12 μM GSK3β inhibitor CHIR99021 (CHIR) (Selleckchem) and 0.6 mM of L-ascorbic acid 2-phosphate (AA) (Sigma-Aldrich, USA), followed by cultivation in RPMI+B27+IN− supplemented with 2.5 μM tankyrase inhibitor IWR-1 (Selleckchem, USA). Beating MC-iPSCs harvested on day 14-15 were trypsinized into single cells and subsequently subjected to flow cytometry analysis for the quantification of the expression of Troponin T (cTnT) as described above.

### Erythroblast Differentiation

Erythroblast differentiation was done using a BMP4-based protocol (Sivalingam, Chen et al. 2018). Briefly, aggregates (used as EBs) of MC-iPSCs (1×10⁶ cells/mL) were cultivated in 5 ml of StemLine® II Hematopoietic Stem Cell Expansion medium (SL2; Sigma-Aldrich) supplemented with BMP4 (R&D system), VEGF (Peprotech), Activin A (StemCell Technologies) and CHIR for 1 day under agitation (75 rpm). On day 2, CHIR was removed by adding fresh SL2 with BMP4, VEGF, Activin A, and β-estradiol. On day 3 of differentiation, a single-cell suspension was harvested from the cell-MC aggregates using 1× TrypLE™ Express as described above. Single cells (2.5×105 cells/ml) were then replated in SL2 supplemented with BMP4, VEGF, bFGF, SCF, IGF2 (StemCell Technologies), TPO (Peprotech), Heparin (Sigma), 3-isobutyl-1-methylxamthine (IBMX; Sigma) and β-estradiol for hematopoietic induction, with medium changes every alternative day. On day 11, cells were harvested and analyzed for the expression of DRAQ5 (Deep Red Anthraquinone 5) by flow cytometry.

### HSC Differentiation

HSC differentiation, using BMP4 based protocol has previously been described (Sivalingam, Chen et al. 2018), which is similar to the aforementioned erythroblasts differentiation protocol with minor modification. Briefly, differentiation was initiated in MC cultures by media change to SL2 supplemented with BMP4, VEGF, Activin A, and CHIR. Thereafter, daily medium changes to SL2 supplemented with different cytokines were carried out as described in Sivalingam's report. On day 3 of differentiation, single cells were derived from the cell-MC aggregates following treatment with TrypLE™ Express followed by straining the MC through 40 μm cell strainers. Single cells in suspension were seeded in SL2 supplemented with BMP4, VEGF, bFGF, SCF, IGF2, TPO, Heparin, 3-isobutyl-1-methylxamthine and β-estradiol for hematopoietic induction until Day 11. HSC differentiation was evaluated by the percentage of CD34+/CD43+ and CD34+/CD45+ cells.

### Statistical Analysis

Unless otherwise specified, all statistical data is the result of 3 independent experimental repeats, in which numbers were collected independently at the conclusion of the experiment. All data are expressed as the mean±SEM using the statistical software GraphPad Prism®, version 4.1. Statistical significance was determined from at least 3 independent experiments. Comparisons of two data sets were statistically analyzed with Student's t-test. Multiple comparisons between more than three groups were performed using analysis of variance with one-way ANOVA multiple comparison tests. A p-value is shown when the difference between compared groups is significant, with p<0.01, p<0.001, p<0.001, and p<0.0001 considered levels of statistically significant differences.

### Results

### Example 1: Common Technique for iPSCs Reprogramming

The inventors had used human lung fibroblast IMR90 cells as example for conventional reprogramming approach on monolayer (MNL) cultures (FIG. 2). The IMR90 fibroblasts were seeded on tissue culture plates and were transduced by Sendai virus expressing Oct4, Sox2, c-Myc, and Klf4. After transduction, cells were daily checked for morphological changes and these became visible by means of colony formation (about 7 days). Due to the area limitation of the tissue culture plates, only 5-10 colonies were identified and picked (about 15 days). Those iPSCs colonies were selected on the basis of hESC-like morphology by manual picking. The picked colonies were then allowed to further expand on laminin-coated tissue culture plates (about 23 days) and then frozen down for banking (about 31 days).

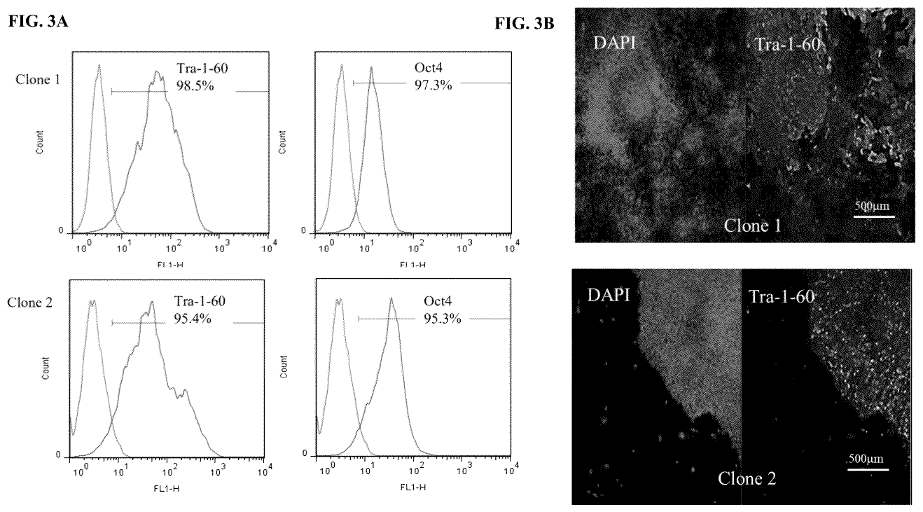

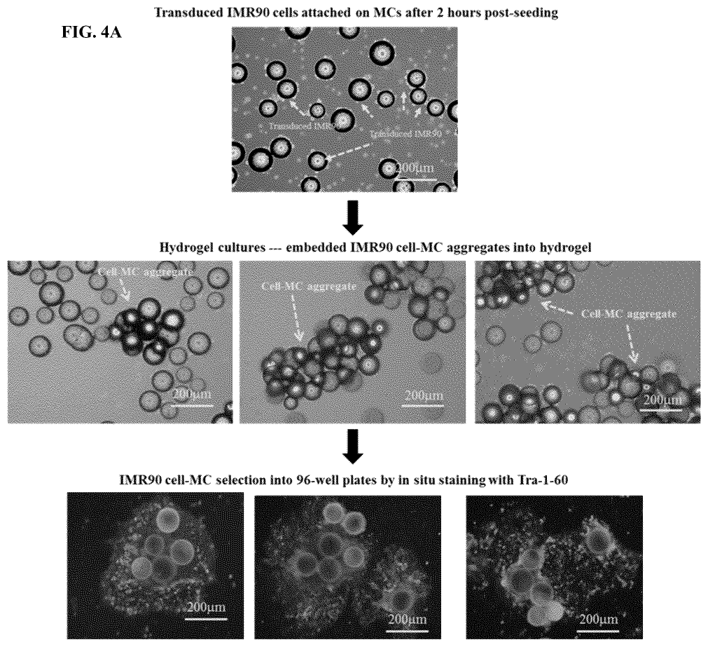

To confirm the pluripotency of the picked colonies, cells were trypsinized and replated on 0.1% (w/v) gelatinized plates for immunocytochemistry staining of surface marker Tra-1-60 (FIG. 3(B)) and FACS analysis of Tra-1-60 and transcription factor Oct4 (FIG. 3(A)).

### Example 2: iPSC Generation by Conventional Method in MNL Cultures

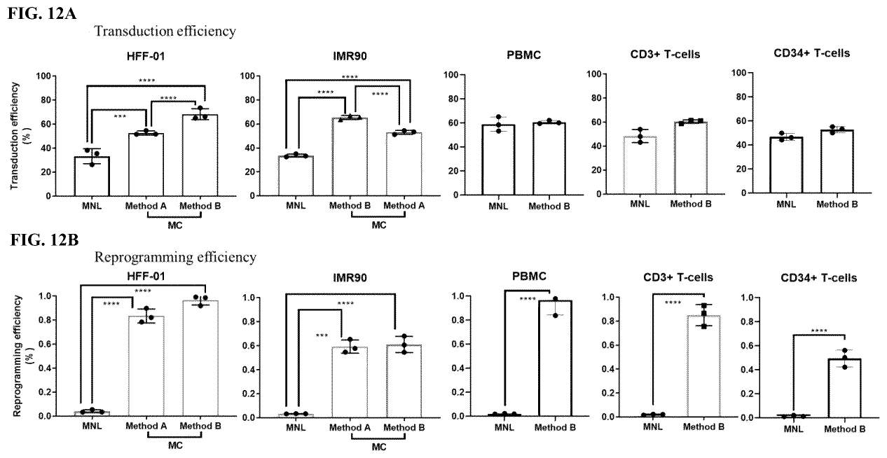

Adherent fibroblasts HFF-01, IMR90, suspension PBMC, CD3+ T-cells and CD34+ cells were transduced with Sendai virus reprogramming factors using the conventional MNL method following the manufacturer's instructions (ThermoFisher Scientific), except for LN521 being used as an adhesive substrate rather than vitronectin. iPSC-like colonies (designated as MNL-iPSCs) began appearing at day 12 post-transduction. Reprogramming efficiencies of HFF-01, IMR90, PBMC, CD3+ T cells, and CD34+ cells were 0.04±0.02%, 0.03±0.001%, 0.02±0.004%, 0.02±0.0001%, and 0.015±0.008%, respectively, as calculated by the number of Tra-1-60+ colonies obtained on day 14 per initial cell seeding (Table 2 & FIG. 12(B)).

TABLE 2
Summary table of transduction efficiency, reprogramming efficiency, and number of Tra-1-60+ clones per well
emerged at day 14 after reprogramming. The reprogramming efficiency was calculated by dividing the number of
resulting Tra-1-60+ clones at day 14 by the number of input cells and multiplying by 100%. Mean ± SEM. (n = 3).

| | Transduction efficiency (%) | | | Reprogramming efficiency (%) | | | No. of Tra-1-60+ cells/well | | |
|---|---|---|---|---|---|---|---|---|---|
| | | MC | | | MC | | | MC | |
| | | Method | Method | | Method | Method | | Method | Method |
| | MNL | A | B | MNL | A | B | MNL | A | B |
| HFF-01 | 33.4 ± 6.3 | 52.7 ± 1.7 | 68.4 ± 4.6 | 0.04 ± 0.02 | 0.84 ± 0.1 | 0.97 ± 0.003 | 40 ± 4 | 220 ± 10 | 331 ± 30 |
| IMR90 | 33.7 ± 1.3 | 53.1 ± 1.6 | 65.5 ± 1.7 | 0.03 ± 0.001 | 0.59 ± 0.1 | 0.61 ± 0.04 | 34 ± 1 | 157 ± 11 | 201 ± 25 |
| PBMC | 59.1 ± 5.9 | N.A. | 60.7 ± 1.4 | 0.02 ± 0.004 | N.A. | 0.97 ± 0.1 | 36 ± 2 | N.A. | 293 ± 31 |
| CD3+ | 48.5 ± 5.5 | N.A. | 60.5 ± 1.4 | 0.02 ± 0.001 | N.A. | 0.85 ± 0.05 | 30 ± 7 | N.A. | 257 ± 22 |
| T cells | | | | | | | | | |
| CD34+ | 46.9 ± 2.9 | N.A. | 52.9 ± 2.6 | 0.015 ± 0.008 | N.A. | 0.50 ± 0.1 | 20 ± 8 | N.A. | 131 ± 16 |
| cells | | | | | | | | | |
| 3F- | 36.9 ± 1.9 | N.A. | 69.4 ± 6.0 | 0.0009 ± 0.004 | N.A. | 0.15 ± 0.03 | 3 ± 1 | N.A. | 77 ± 16 |
| HFF01 | | | | | | | | | |

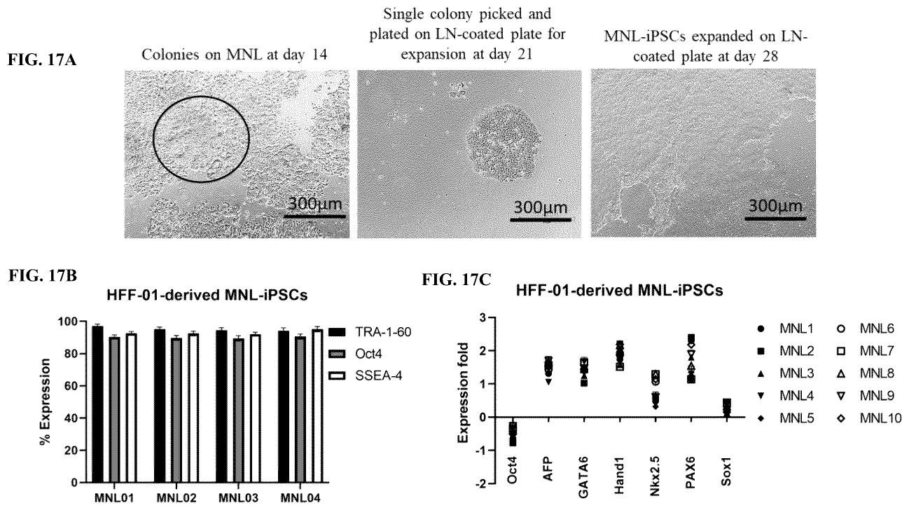

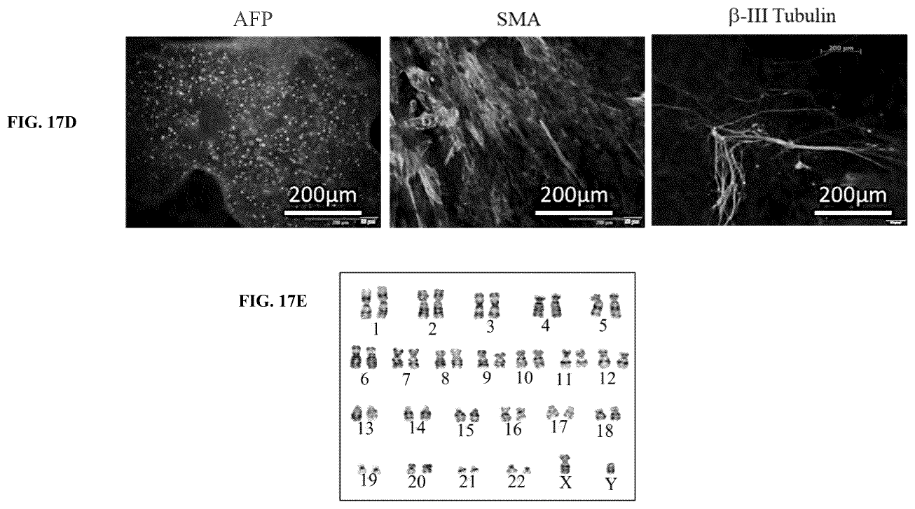

Four MNL-iPSC colonies randomly isolated (FIG. 17(A)), were passaged 3-4 times in order to obtain enough amount of cells for analysis. The cells were analyzed for expression of Oct4, Tra-1-60, and SSEA4 (FIG. 17(B)), RT-qPCR analysis of expression of differentiation-associated genes (FIG. 17(C)), spontaneous differentiation (FIG. 17(D)) and karyotype (FIG. 17(E)). Results shows that that all lines tested are pluripotent, have the capacity to differentiate into the three germ layers and have diploid karyotype.

However, this is an inefficient process taking an average of 6-8 weeks before a limited number of colonies are established, and cell generation is sufficient for characterization. Technical limitations abound, such as time-consuming passaging by enzymatic cell dissociation and the difficulty of testing the differentiation efficiency in EBs culture in the mid-phase of cell line development. To overcome these challenges, the inventors had set out to design a more efficient reprogramming platform using MCs, named ReprograMC.

### Example 3: MCs Based iPSCs Reprogramming (ReprogaMC)

Since somatic cells reprogramming and the subsequent verification of iPSCs pluripotency are laborious, manual processes, this is a limit to the scale and reproducibility of this process. A MCs platform for iPSCs reprogramming enabling automated, high-throughput conversion of somatic cells into iPSCs and their differentiation with minimal manual intervention is developed (FIG. 1(B)). The inventor had used HFF-01, human lung fibroblast IMR90, PBMC, T-cells from human finger-pricked blood, and human cord blood CD34+HSCs as examples for the MCs-based reprogramming platform.

After transduction of the HFF-01, IMR90 fibroblasts, PBMC, CD3+ T-cells, and CD34+HSCs by Sendai virus expressing reprogramming factors, the transduced cells were seeded on MCs (laminin-521 coated MCs) allowing the process of reprogramming to take place (FIG. 1(B), Method B). On the other hand, transduction can also be done on MCs pre-seeded with cells (FIG. 1(B), Method A). Cell-MC aggregates became visible after 3-5 days post-transduction in both indirect and direct MCs reprogramming approaches.

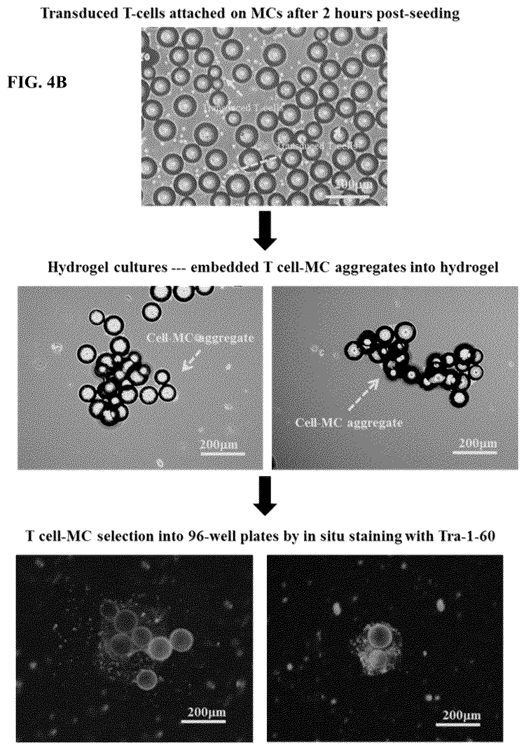

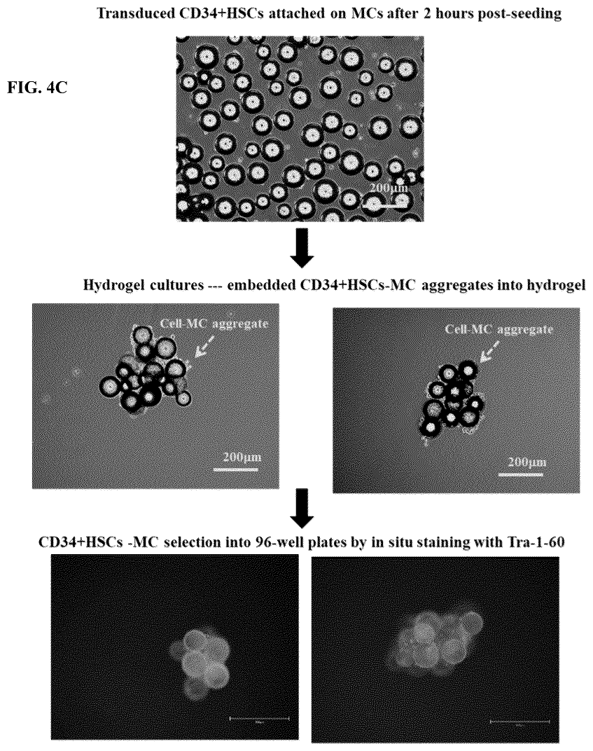

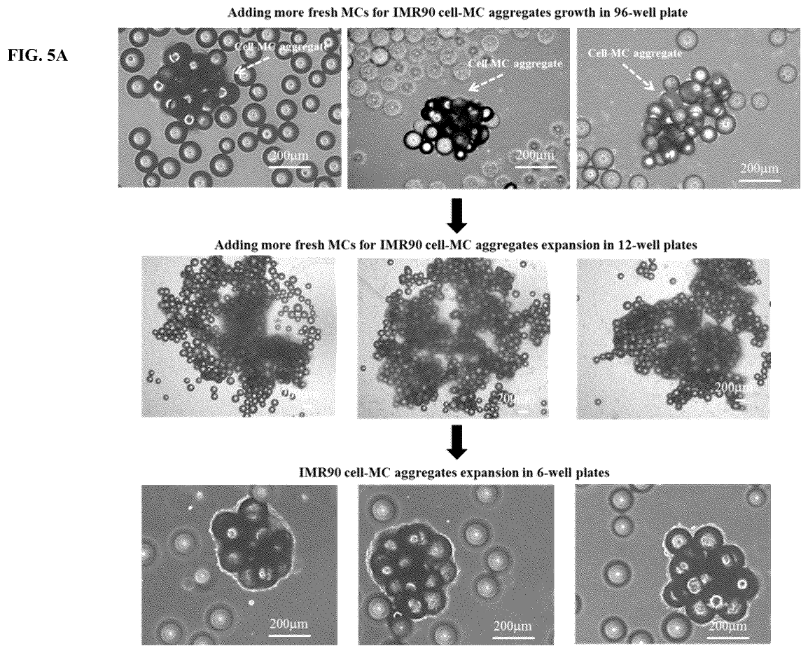

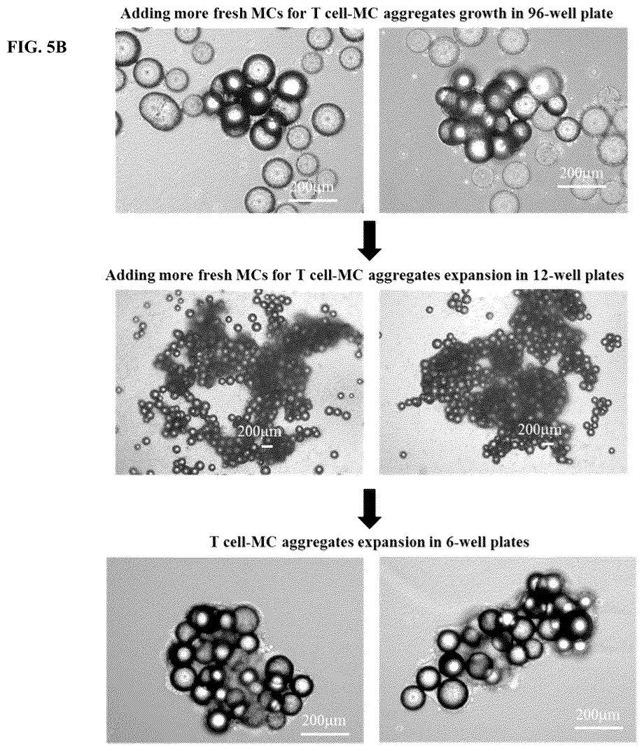

Transduced HFF-01, IMR90, PBMC, CD3+ T-cells, and CD34+HSCs-MC (cell-MC) aggregates of the MCs cultures were then immobilized in 0.5% (w/v) agarose gel for easy aggregates monitoring, screening, and picking. After 7 days cultures, immunocytochemistry staining of surface marker Tra-1-60 was used to identify the pluripotency of all cell-MC aggregates in the hydrogel (for examples, IMR90: FIG. 4(A); T-cells: FIG. 4(B); CD34+HSCs: FIG. 4(C)). Those Tra-1-60⁺ cell-MC aggregates were picked and transferred to 96-well plates (for examples, IMR90: FIG. 5(A); T-cells: FIG. 5(B); CD34+HSCs: FIG. 5(C)). Up to 96 cell-MC aggregates can be picked and handling in a simple screening process. An integrated robot system can be introduced to greatly increase the number of evaluations, and reduce the time scale of reprogramming and the labor involved. The aggregates were allowed for further propagation and expansion by adding more freshly prepared MCs. After another 7 days cultures, only the fast-growing iPSCs aggregates (according to their size changes) were selected and transferred to 12-well plates for further expansion by again adding more freshly prepared MCs (for examples, IMR90: FIG. 5(A); T-cells: FIG. 5(B); CD34+HSCs: FIG. 5(C)). After another 7 days, the fast-growing iPSCs aggregates in 12-well plates were sub-cultured again into 6-well plate by adding more freshly prepared MCs (for examples, IMR90: FIG. 5(A); T-cells: FIG. 5(B); CD34+HSCs: FIG. 5(C)).

After expansion on 6-well plates, samples of iPSCs-like cell-MC clones were subjected to characterization.

### Example 4: Characterization of MC-iPSCs

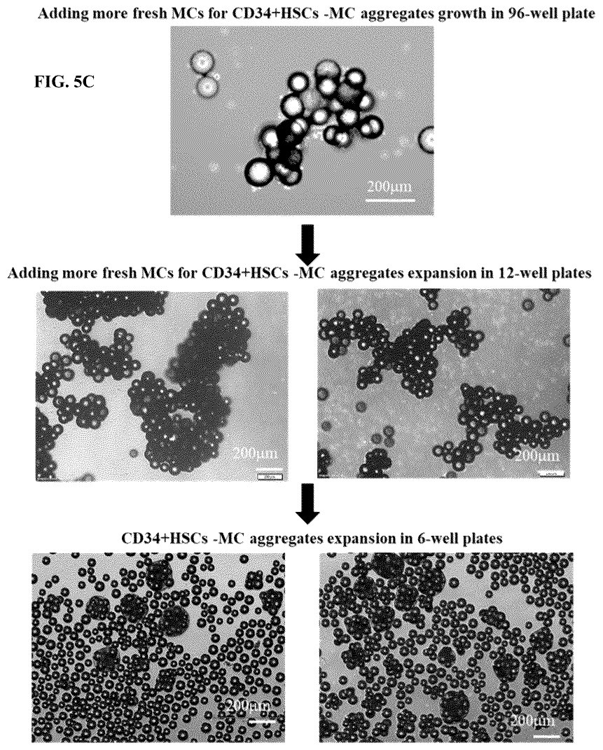

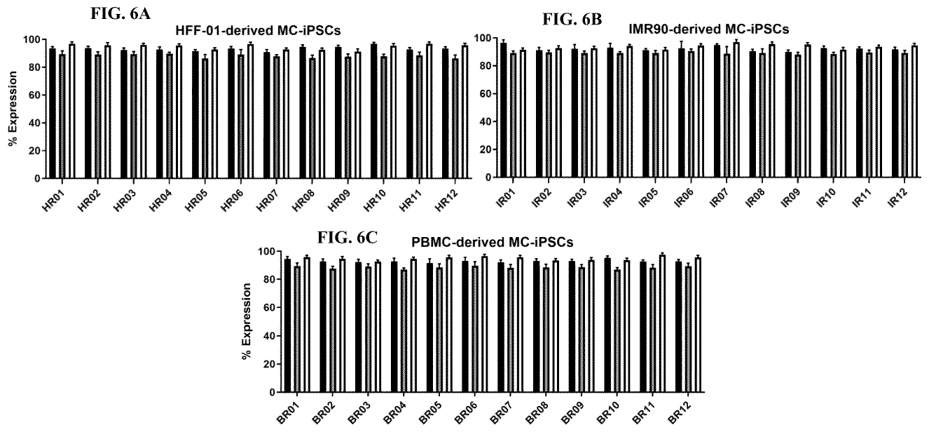

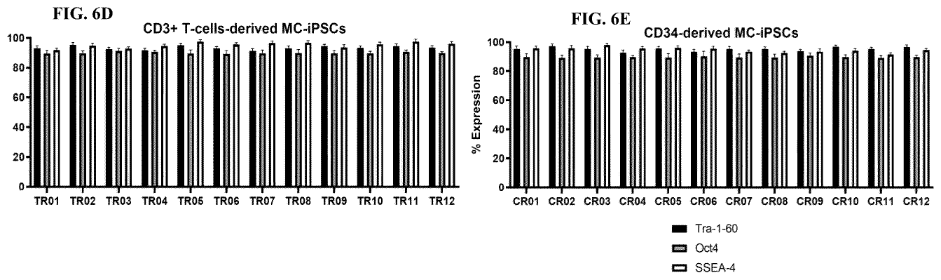

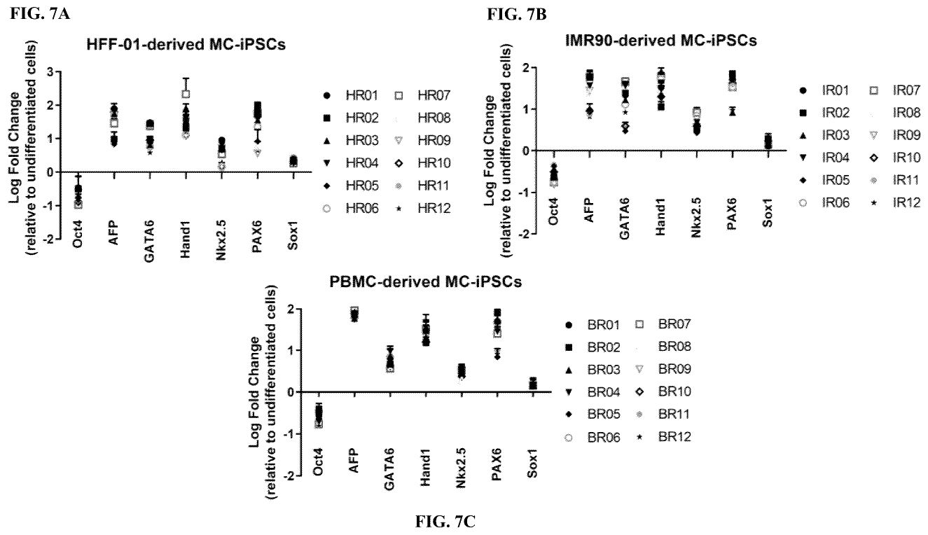

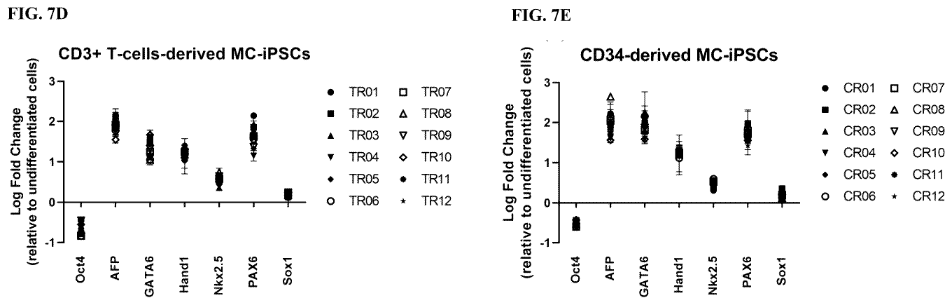

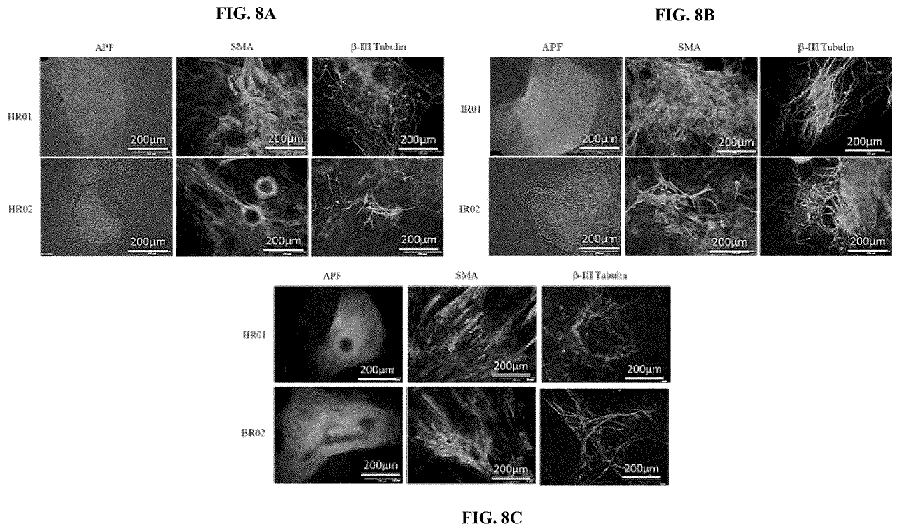

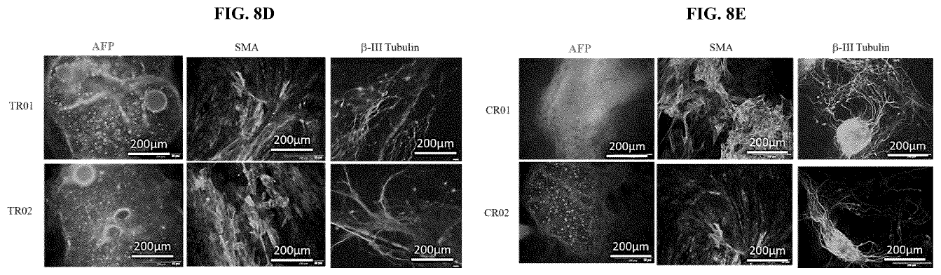

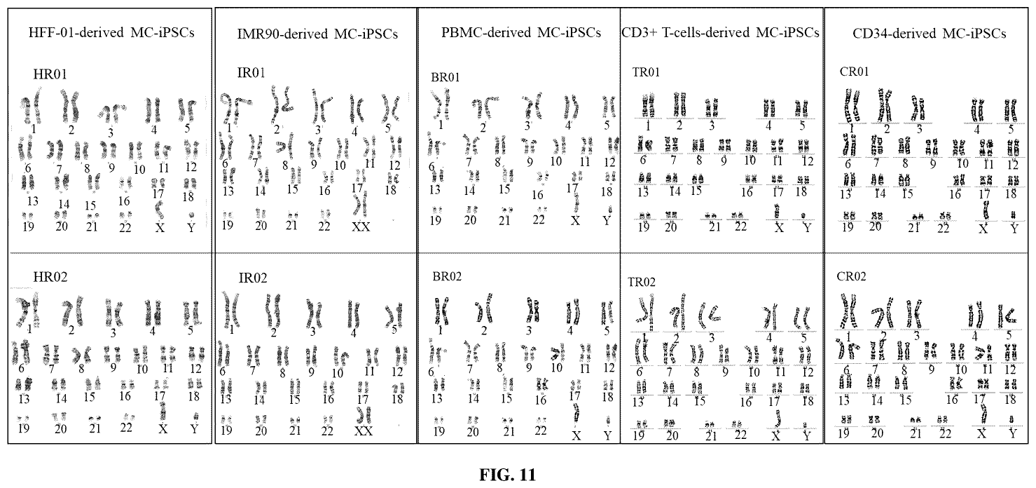

Following ReprograMC reprogramming, 60 MC-iPSCs from 5 somatic origin: HFF-01, IMR90, PBMC, CD3+ T-cells, and CD34+ cells (12 clones of each source) were further characterized. The cells were analyzed for expression of Oct4, Tra-1-60, and SSEA-4 (FIG. 6), RT-qPCR analysis of expression of differentiation-associated genes (FIG. 7), spontaneous differentiation (FIG. 8) and karyotype (FIG. 11). Results shows that all lines tested are pluripotent, have the capacity to differentiate into the three germ layers and have diploid karyotype.

It is noteworthy that variation in gene expression levels between different samples and cell sources were observed during analysis of expression of differentiation-associated genes. For instance, all IMR90-derived MC-iPSCs expressed more germ layer markers (FIG. 7(B)), whereas all CD3+ T-cells and CD34-cell-derived MC-iPSCs only highly expressed mesoderm markers, with other markers expressed to a lesser degree (FIG. 7(D) and FIG. 7(E)). HFF-01-derived MC-iPSCs expressed the endoderm marker AFP and GATA6 to a higher extent than other markers (FIG. 7(A)), indicating that they may be more favorable for differentiation into cells of the endodermal lineage.

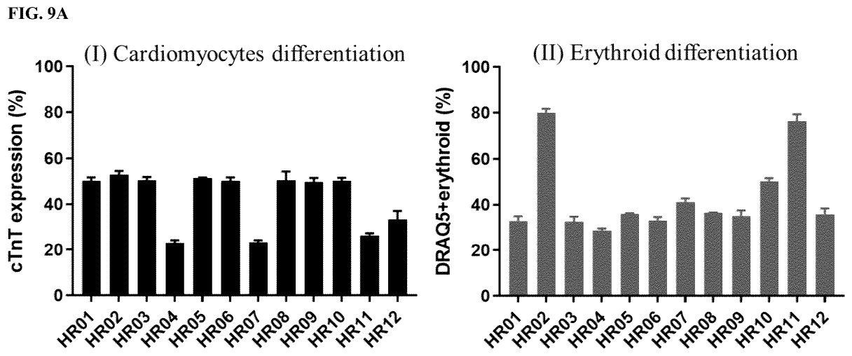

To further confirm the development potential of the MC-iPSCs, a fraction of 12 HFF-01-derived MC-iPSCs and 12 IMR90-derived MC-iPSCs suspension cultures was collected and transferred to differentiation medium for cardiomyocyte differentiation following the published protocol (Lam, Chen et al. 2014). It is worth noting that although all the differentiated HFF-01-derived MC-iPSCs and IMR90-derived MC-iPSCs examined were positive for expression of the cardiomyocyte marker cTnT, both cells sources have 4 clones that exhibited lower levels of cTnT (<30%) than other aggregates (˜60%) (FIG. 9).

The inventors had also compared the erythroblast differentiation potential of the 12 HFF-01-derived iPSCs and 12 IMR90-derived iPSCs following the published blood differentiation protocols (Sivalingam, Chen et al. 2018). Erythroblasts clones were functional and has oxygen carrying ability (data not shown). Although all could differentiate to erythroblasts, the erythroid potential varied between clones with 2 expressing DRAQ5 (a marker of erythroblast) to a high degree (>80%; FIG. 9). As concluded, cell line-to-cell line variation may occur even if they are derived from the same source. Higher number of iPSC clones generated from the MC based platform described herein provides higher chance to find the best clone for cell differentiation.

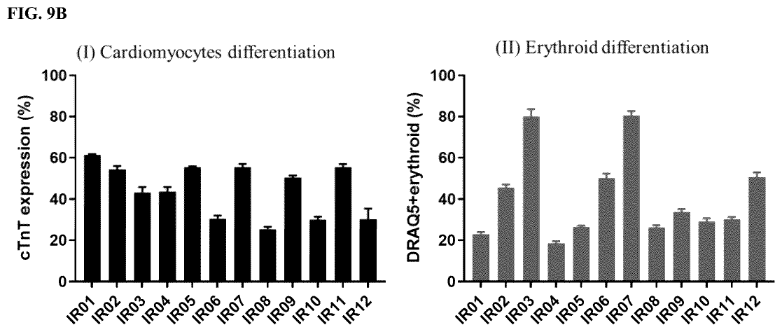

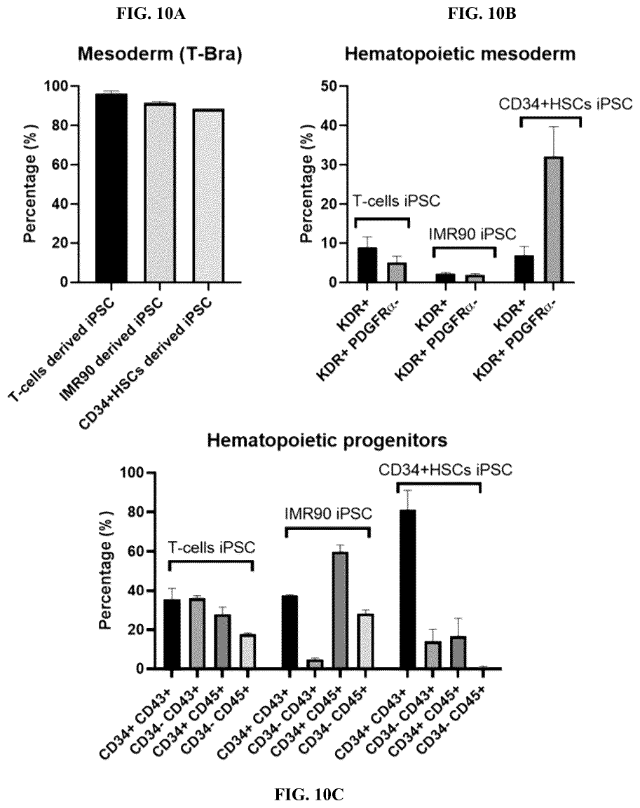

The inventors had also proceeded to evaluate hematopoietic progenitor cells differentiation of IMR90, T-cells−, and CD34+HSC-derived iPSC-MC clones. Cells on MC were differentiated into T-Bra+ primitive streak/mesoderm (>90%) on day 1 of differentiation (FIG. 10(A)) and had evidence of hematopoietic fated mesoderm marker KDR+PDGFRα− (2-12%) by day 3 of differentiation (FIG. 10(B)). By day 12 of differentiation, CD34+/CD45+ (30-60%) and CD34+/CD43+ (40-80%) hematopoietic progenitor cells and more mature CD34−/CD45+ (20-30%) and CD34−/CD43+ (6-38%) hematopoietic committed cells were detected in all the differentiated cultures (FIG. 10(C)). iPSC-derived from cord blood CD34+ expressed highest percentage of CD34+/CD43+ hematopoietic progenitor cells; however, iPSC-derived from IMR90 on MC expressed the highest percentage of CD34+/CD45+ hematopoietic progenitor cells. As concluded, cell line-to-cell line variation may occur, and again higher number of iPSC clones generated from the MC based platform provides higher chance to find the best clone for cell differentiation.

### Example 5: Reprogramming by ReprograMC Approach Enhances Transduction and Reprogramming Efficiencies

Two agitated ReprograMC approaches were explored (FIG. 1(B)): ReprograMC A (transduction after cells attached and spread on the surface of the MCs) and ReprograMC B (transduction at early suspension state before seeding on MCs). For each MC reprogramming run, 20 mg of MCs was used, resulting in a total available surface area of ˜10 cm², comparable to a single well of a 6-well plate for MNL cultures.

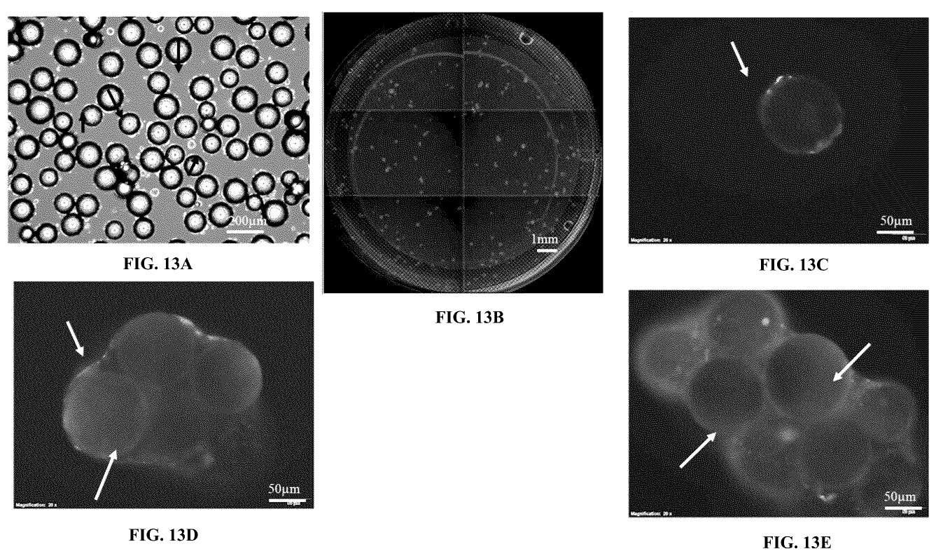

ReprograMC A: The inventors had attempted to reprogram HFF-01 and IMR90 fibroblasts which were initially attached and spread on the surface of MCs for 2 days in agitated cultures (FIG. 13(A)). Higher transduction efficiency was observed (HFF-01: 52.7±1.7% and IMR90: 53.1±1.6%) when compared with the conventional MNL method (HFF-01:33.4±6.3% and IMR90: 33.7±1.3%; p<0.01) (Table 2 & FIG. 12(A)). Most importantly, reprogramming efficiency of ReprograMC A (HFF-01: 0.84±0.1% and IMR90: 0.59±0.1%) was ˜20-fold increase over the MNL method (HFF-01: 0.04±0.02% and IMR90: 0.03±0.001%; p<0.00001) (Table 2 & FIG. 12(B)). FIG. 13(B) shows an example of the microscopic view of a well of a 6-well plate with immobilized HFF-01 cell-MC in the TGP hydrogel taken in the ClonePix™ System, at day 14. Gray dots are the Tra-1-60+ cell-MC. For HFF-01, by day 14, about 220±10 Tra-1-60+ cell-MC per well were obtained, compared to only about 40±4 Tra-1-60+ colonies per well from the MNL method, p<0.00001 (Table 2); for IMR90, by day 14, the inventors obtained roughly 157±11 Tra-1-60+ cell-MC per well using Method A, compared to 34±1 Tra-1-60+ colonies per well using the MNL approach, p<0.00001 (Table 2). Single Tra-1-60+ cell-MC embedded in the TGP hydrogel (FIG. 13(C)) were then randomly picked in the ClonePix™ System and transferred in a separate well of a 96 ULA plate containing 0.5 mg LN-coated MCs. Thereafter, subsequent cell expansion and passaging, from 96-well ULA to 12-well ULA (FIG. 13(D)) to 6-well ULA (FIG. 13(E)), were done by simply picking and transferring a fraction of cell-MC aggregates to new LN-coated MCs, without the need of trypsinization. The expanded Tra-1-60+ cell-aggregates in 6-well ULA culture (FIG. 13(E)) were designated as MC-iPSCs. It is important to note that in the MC method the use of cell dissociation solutions or cell scraping is not required for passaging. Moreover, differentiation of the cell-MC aggregates was also simply done by sampling a fraction of aggregates and transferring to differentiation medium, without replating and cell dissociation for EBs formation.

ReprograMC B: The inventors attempted to transduce cells in suspension prior to adding MCs. About 331±30 Tra-1-60+ cell-MC per well were obtained with HFF-01 by day 14 using ReprograMC B (Table 2). Transduction efficiency of ReprograMC B—68.4±4.6% was comparable to ReprograMC A—52.7±1.7%; p=0.05. Reprogramming efficiency of ReprograMC B—0.97±0.003% was comparable to ReprograMC A—0.83±0.1%; p=0.05 (FIG. 12(A) & (B)). It is noteworthy that both transduction and reprogramming from these methods were dramatically better (˜23-fold) than MNL conditions (Table 2 & FIG. 12). Similar results were obtained for IMR90, with comparable differences observed between ReprograMC B and ReprograMC A with respect to the reprogramming (ReprograMC B—0.61±0.04% vs ReprograMC A—0.59±0.1%; p=0.05) and transduction efficiency (ReprograMC B—65.5±1.7% vs ReprograMC A—53.1±1.6%; p=0.05). Likewise, MNL controls were drastically lower (Table 2 & FIG. 12).

After successfully reprogramming fibroblasts into a pluripotent state using ReprograMC B. the inventors attempted to reprogram suspension blood cells (PBMC, CD3+ T-cells, and CD34+ cells) into iPSCs. As above, free suspension cells were first transduced with SeV under agitation (100-110 rpm) for 1 day followed by seeding on MCs. By day 14, 293±31 Tra-1-60+ cell-MC per well were obtained from PBMC, 257±22 from CD3+ T cells, and 131±16 from CD34+ cells, while the MNL method only gave rise to roughly 20 to 36 Tra-1-60+ colonies per well from the blood cells within the same period (Table 2). This demonstrated that ReprograMC B exhibits higher reprogramming efficiencies when compared with the MNL method (PBMC: ReprograMC B—0.97±0.1% vs MNL—0.02±0.004%; CD3+ T-cells: ReprograMC B—0.85±0.05% vs MNL—0.02±0.001%; CD34+ cells: ReprograMC B—0.50±0.1% vs MNL—0.015±0.008%; overall p<0.00001, FIG. 12(B) & Table 2). The reprogramming efficiency of ReprograMC B for suspension blood cells was ˜40-fold increase over the MNL method (Table 2 & FIG. 12(B)). However, it is worth noting that there were no significant differences with respect to transduction efficiency between ReprograMC B and MNL (p=0.02) for all mentioned blood cells (FIG. 12(A) & Table 2).

ReprograMC A differs from ReprograMC B in that it requires the growth of the cells on MCs 2 days prior to transduction.

### Example 6: MCs Promote iPSC Generation by Facilitating Gene Activation Early in Reprogramming

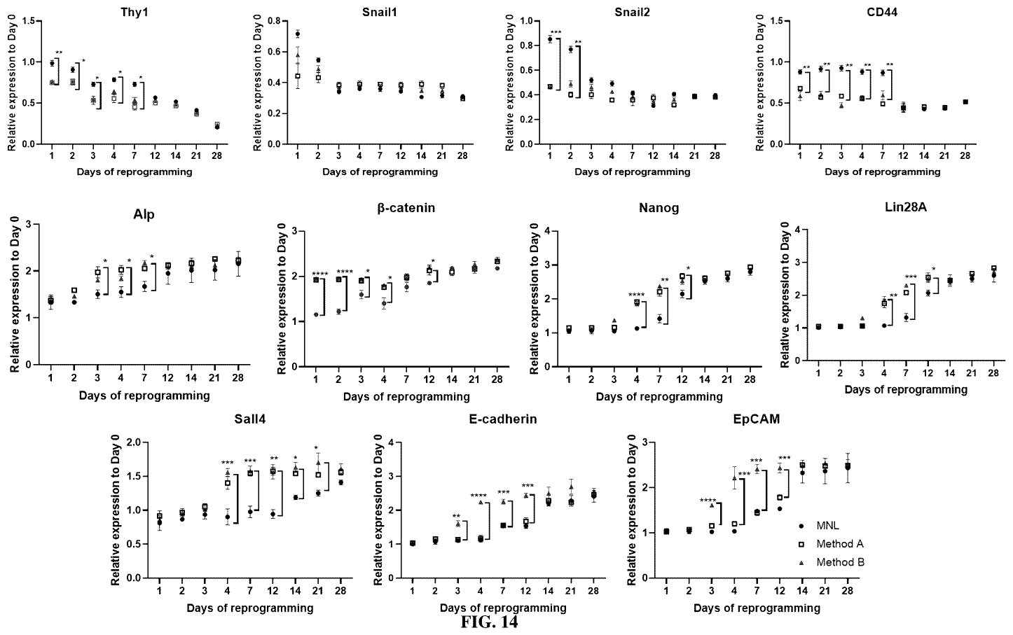

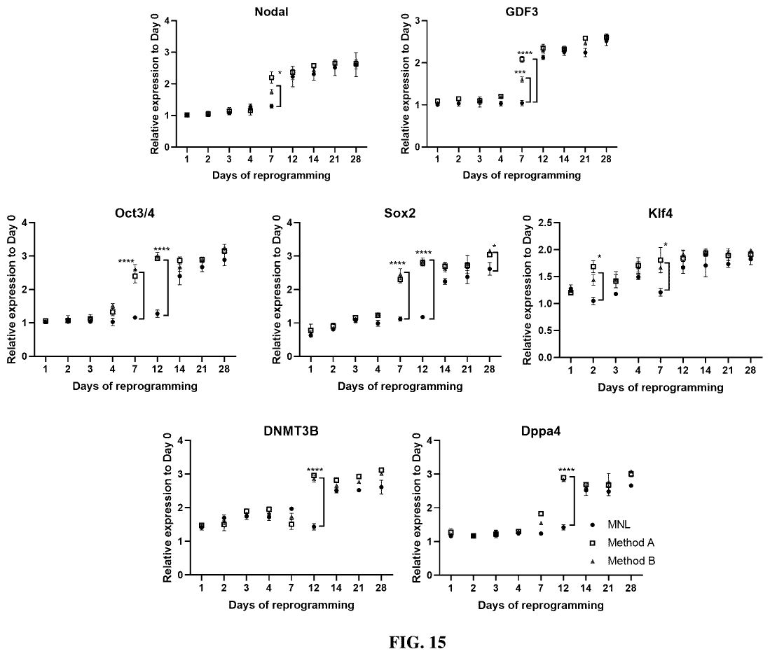

To understand the effect of agitated MC cultures on reprogramming progression, the inventors had performed qPCR studies on genes commonly expressed during the phases of reprogramming using cells harvested at discrete stages of reprogramming (days 0, 1, 2, 3, 4, 5, 7, 14 and 21) from both MNL and ReprograMC A & B cultures. The qPCR results for all reprogramming approaches correlate well with the known sequential molecular events in human fibroblasts: (1) Initiation phase: downregulation of the fibroblast-specific surface markers (such as Thy1 and CD44), coupled with a loss of mesenchymal cell signature (such as Snail1/2), and particularly induction of the signal transducer β-catenin and Alkaline Phosphatase (Alp) (FIG. 14); (2) Maturation phase: upregulation of endogenous Nanog and Lin28, Wnt effector Sall4, epithelial genes EpCAM, and E-cadherin (FIG. 14); and, finally (3) Stabilization phase: acquisition of full pluripotency signature such as expression of endogenous Oct4 and Sox2, Klf4, Nodal, GDF3, DNMT3B and developmental pluripotency associated 4 (DPPA4) (FIG. 15).

Interestingly, the inventors had found that the ReprograMC A culture accelerated the early expression of β-catenin on day 1, at least 2 days earlier than was observed in the MNL method (FIG. 14). Nanog, Lin28a, and Sall4 were also expressed as early as day 4, compared to MNL culture which only reached equivalent levels at day 12 or later (FIG. 14). An early and high induction of E-cadherin and EpCAM was also observed in ReprograMC B on day 3 (FIG. 14). These data suggest that the ReprograMC cultures accelerate MC-iPSC generation through an earlier induction of the epithelial-mesenchymal transition (EMT) and mesenchymal-epithelial transition (MET) process.

In summary, the inventors have demonstrated the agitated MC-based platform, ReprograMC, for the generation of iPSCs from fibroblasts and blood cells that shows higher reprogramming efficiency compared to traditional MNL methods. The inventors had confirmed MC-iPSC clones exhibited high levels of pluripotency and maintained their differentiation potential for all three germ layers as well as the differentiation to cardiomyocytes and blood lineages.

### Example 7: Expedited Derivation of 3F-MC-iPSCs (OKS) in ReprograMC Cultures

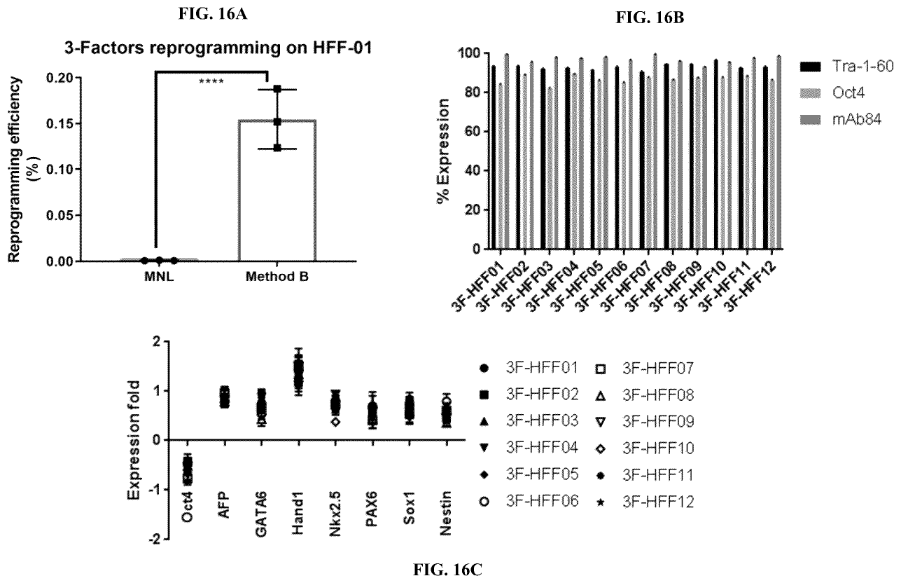

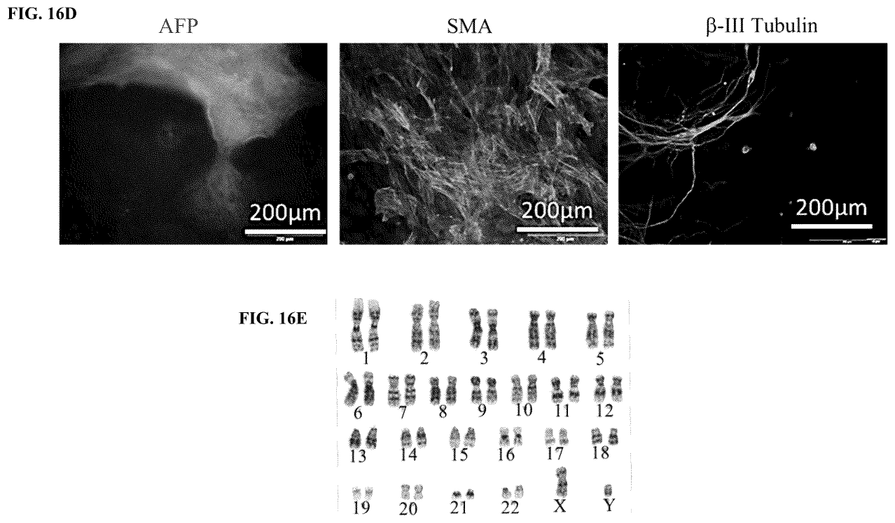

The inventors had tested ReprograMC B reprogramming with three factors: Oct4, Sox2, and Klf4 (c-Myc being eliminated) using MNL culture reprogramming as control. In comparison, while reprogramming efficiency was 0.97±0.003% with 4 factors, efficiency was 0.15±0.03% with 3 factors (FIG. 12(B), FIG. 16(A) and Table 2). The number of Tra-1-60+ cells obtained using 3F transduction is higher in ReprograMC B compared with MNL method (77±16 vs 3±1, Table 2). However, reprogramming efficiency was 166-fold higher in ReprograMC B vs MNL system (0.15% vs 0.0009%, FIG. 16(A) & Table 2).

Twelve clones originated from 3 factors transduction were analyzed for expression of Oct4, Tra-1-60, and mAb84 (FIG. 16(B)), RT-qPCR analysis of expression of differentiation-associated genes (FIG. 16(C)), spontaneous differentiation (FIG. 16(D)), and karyotype (FIG. 16(E)). Results shows that all lines tested are pluripotent, have the capacity to differentiate into the three germ layers and have diploid karyotype.

### Discussion

MC cultures are favorable for maintaining stem cell proliferation and differentiation, and are characterized by a high surface-to-volume ratio which allows for high density cell culture. Utilizing the full potential of MC cultures could help simplify the process of deriving and expanding iPSCs for therapeutic applications, offering a robust and scalable suspension platform for large-scale generation of clinical grade iPSCs.

The inventors had examined whether MC cultures provide a selective advantage to enhance iPSC reprogramming and selected for iPSC with efficient differentiation abilities. The inventors had demonstrated that suspension MC cultures with agitation significantly improved the reprogramming efficiency from both human adherent and suspension somatic cells. The resulting MC-iPSCs possess pluripotency and robust differentiation characteristics and display a normal karyotype. By applying this approach to somatic fibroblasts, as well as peripheral blood mononuclear cell (PBMC), CD3+ T-cells, and CD34+ hematopoietic progenitor cells, hundreds of fully reprogrammed iPSCs can be derived, providing ˜50-fold more clones/candidate iPSCs than conventional adherent culture methods. The resulting microcarrier-derived iPSCs (MC-iPSCs) resemble embryonic stem cells in their in vitro characteristics, including gene expression and differentiation potential. This MC reprogramming approach has the added potential to enhance other areas of iPSC research such as CRISPR edited clone selection.

The inventors had also investigated if iPSC with c-Myc elimination could still be derived at high efficiency on the MC platform.

Using a conventional monolayer process includes multiple steps of cell expansion, dissociation, phenotype evaluation, banking and finally differentiation towards target functional cells. Reprogramming cells from only a few samples from a single patient requires a full-time dedicated expert over a costly 2-3-month period. Recently, scientists have attempted to develop more efficient systems that allow for high-throughput generation of iPSCs for industrial or clinical use. However, these still rely on conventional monolayer reprogramming and selection, and are thus relatively slow, inefficient and with high demands for space and manpower. Overcoming these challenges will rapidly push the iPSC field towards safer and more scalable reprogramming methods.

In the present disclosure, the inventors have utilized an agitated MC suspension platform, ReprograMC, using 5 sources of human adherent and suspension somatic cells, to enhance the reprogramming efficiency by approximately 20- to 50-fold compared to conventional static MNL platforms (FIG. 12(B)). The resulting MC-iPSCs possess pluripotency, high differentiation potential, and display a normal karyotype. This novel MC reprogramming approach has the potential to streamline the iPSC manufacturing process from cellular reprogramming, iPSC expansion, quality assurance, master/working cell banking and directed differentiation to a relevant functional cell type without time-consuming and laborious processes such as single cell dissociation for subculturing followed by re-aggregation on separate plates as EBs.

### Differential Expression of Reprogramming Related Genes Among Different Approaches

In order to reveal the main impact of agitated MC culture on reprogramming, the inventors had compared a set of known genes found in literature commonly associated with the three phases of reprogramming: initiation, maturation, and stabilization, between the static MNL culture and the agitated MC culture (Methods A and B).

FIGS. 14 and 15 show that an early induction of β-catenin on day 1, Nanog, Lin28A, and Sall4 on day 4 were observed in agitated MC Method A (transduction after cells attached and spread on the surface of the MCs), compared to the static MNL culture. Oct4, Sox2, and Klf4 were also upregulated on day 7. The inventors hypothesized that agitated MC culture-induced early and high expression of β-catenin may enhance the expression of pluripotency circuitry genes, through an interaction with Klf4, Oct4 and Sox2, to promote cell reprogramming or enhance Oct-4 activity and consequently reinforce pluripotency.

Notably, the higher expression of β-catenin could also activate the canonical Wnt signaling pathway. The effects of Wnt/β-catenin signaling activity on different stages of reprogramming has previously been reported with activation of Wnt signaling during the initiation phase leading to a significant improvement in reprogramming efficiency. There is evidence to suggest that mechanical stress could induce cellular reprogramming through the Wnt/β-catenin signaling pathway. This may also explain the higher reprogramming efficiency without c-Myc in the agitated MC culture versus MNL approaches (FIG. 16(A)) since c-Myc was found to be one of the downstream targets of β-catenin.

The rapid induction of Nanog, Lin28A, and Sall4 in ReprograMC cultures on day 4 (FIG. 14) indicated that the cells entered the maturation phase. much earlier than in the MNL culture, which reached maturation on day 12 or later (FIG. 15). Notably, the maturation phase has been identified as the major roadblock for acquisition of pluripotency in cell reprogramming. The inventors hypothesized that the rapid induction of some of the maturation phase gene markers, Nanog, Lin28A, and Sall4 improved reprogramming efficiency. Additionally, Sall4 has also been reported as a reprogramming enhancer. Studies have shown that transduction of Sall4 gene in mouse and human somatic cells could significantly enhance the efficiency of iPSC generation. The inventors attribute the shear stress generated by the agitation as the major trigger for the transcriptional changes observed in the transition from initiation to maturation phases.

Reprogramming of Single Cell Suspension Further Increases iPSC Generation Efficiency

Overall, the two agitated MC approaches (ReprograMC A and ReprograMC B) showed no notable differences in gene expression patterns. In human cells, the activation of E-cadherin and EpCAM, indicators of the onset of mesenchymal-to-epithelial transition (MET), only occurred at the stages of maturation of reprogramming (beyond the first 3 days of the initiation phase), where the cells acquired pluripotency with endogenous Oct4 activation.

Importantly, the inventors had demonstrated the ReprograMC approach can produce iPSCs with high differentiation potential forming all three germ layers and further demonstrated the formation of functional cardiomyocytes and erythroblasts. It is worth noting that there is variation between the differentiation efficiencies of different clones (FIG. 16) and that a clone that differentiates efficiently to one cell type (e.g., cardiomyocytes) does not necessarily differentiate well to the other type (e.g., erythroblasts). Therefore, analysis using cell-MC aggregates instead of EBs enable high-throughput selection of specific clones for further development.

In conclusion, the inventors demonstrated that ReprograMC approach provide an induction advantage for enhanced iPSC generation. The technology disclosed herein has the potential to accelerate and standardize iPSC research, bringing it to clinical applications more rapidly.

To summarize, reprogramming somatic cells to pluripotent stem cells (iPSCs) by monolayer cultures is plagued by low efficiencies, high levels of manipulation and operator unpredictability during the multistep process of choosing and subculturing a limited number of clones for expansion and differentiation. The inventors have developed an all-in-one ReprograMC (Reprogramming on Microcarriers) method to solve these challenges. To produce clinically useful iPSCs, it is important to choose the correct donor cell type and the best reprogramming method. Human somatic cells, such as skin biopsy (e.g. foreskin fibroblasts) and human blood samples (e.g. erythroblasts, T-cells, and hematopoietic stem cells) are favoured cell types for the induced of pluripotency because they are from the patient own tissue, easy to obtain and easy to reprogram. Five sources of human somatic cells were reprogrammed, selected, expanded, and differentiated by the ReprograMC method. Improvement of transduction efficiencies of up to 2 times was observed using Sendai virus. Accelerated reprogramming by the ReprograMC method was 7 days faster than monolayer, providing between 20 to 50-fold more clones to choose from fibroblasts, peripheral blood mononuclear cells, T cells and CD34+ stem cells. This was observed to be due to an earlier induction of genes (β-catenin, E-cadherin and EpCAM) on day 4 vs. monolayer cultures which occurred on days 14 or later. Higher expression of β-catenin could activate the Wnt/β-catenin pathway which is necessary during the initiation phase of reprogramming. Following that, faster induction, and earlier stabilization of pluripotency genes (Nanog, Lin28A, Sall4, Oct3/4, Sox 2 and Klf4) occurred during the maturation phase of reprogramming (after day 7) in the ReprograMC vs. monolayer method (after days 12-14). Integrated expansion and efficient differentiation to the 3 germ layers as well as to the cardiomyocyte and blood lineages was further demonstrated by the ReprograMC method. This microcarrier, suspension agitated method is also amenable to automation for processing more donor samples in a small footprint; thus, potentially alleviating the many challenges of manual monolayer selection of iPSCs.

Therefore, the present disclosure provides a novel method of iPSCs cell lines production in high numbers by employing automation, high throughput techniques and standardized protocols. The present disclosure also provides a novel microcarriers platform for parallel iPSCs generation, selection, expansion and differentiation towards functional cells (e.g. cardiomyocytes and blood). This provides a platform for large-scale in vitro iPSCs studies. The present disclosure is also the first demonstration that microcarriers technology enables the application of cell reprogramming in the development of personalized medicines. Further, the novel platform described reduces the high complexity of manual processes, which are involved in the production of iPSCs and their differentiated functional cells, in bioprocessing technology.

### REFERENCES

Chen, A. K., X. Chen, et al. (2011). “Critical microcarrier properties affecting the expansion of undifferentiated human embryonic stem cells.” Stem cell research 7(2): 97-111.
Lam, A. T., A. K. Chen, et al. (2014). “Conjoint propagation and differentiation of human embryonic stem cells to cardiomyocytes in a defined microcarrier spinner culture.” Stem cell research & therapy 5(5): 110.
Lam, A. T., J. Li, et al. (2015). “Improved Human Pluripotent Stem Cell Attachment and Spreading on Xeno-Free Laminin-521-Coated Microcarriers Results in Efficient Growth in Agitated Cultures.” BioResearch open access 4(1): 242-257.
Sivalingam, J., H. Y. Chen, et al. (2018). “Improved erythroid differentiation of multiple human pluripotent stem cell lines in microcarrier culture by modulation of Wnt/beta-Catenin signaling.” Haematologica 103(7): e279-e283.

1. A method of reprogramming somatic cells into induced pluripotent stem cells (iPSCs), comprising:
(a) seeding the somatic cells on a plurality of microcarriers (MCs) to form cell-MC aggregates; and
(b) transducing transcription factors into the somatic cells of the cell-MC aggregates;
wherein step (a) and step (b) are carried out under continuous agitation.

2.-24. (canceled)

25. A method of producing, selecting, expanding, characterizing, and differentiating iPSCs, comprising:
carrying out steps (a)-(b) of claim 1;
(c) immobilizing the cell-MC aggregates into a hydrogel;
(d) selecting the cell-MC aggregates showing fast cell growth or expressing pluripotency markers;
(e) expanding the selected cell-MC aggregates by adding fresh MCs to the selected cell-MC aggregates;
(f) characterizing the cell-MC aggregates to determine whether the somatic cells on the MCs have been reprogrammed to iPSCs; and
(g) differentiating the cells of the cell-MC aggregates toward functional cells.

26-34. (canceled)

35. A method of reprogramming somatic cells selected from the group consisting of fibroblasts IMR90, fibroblasts HFF-01, PBMC, CD3+ T cells and CD34+ hematopoietic stem cells (HSCs) into induced pluripotent stem cells (iPSCs), comprising:
(a) seeding the somatic cells on a plurality of microcarriers (MCs) to form cell-MC aggregates;
(b) transducing transcription factors into the somatic cells of the cell-MC aggregates using Sendai virus;
(c) immobilizing the cell-MC aggregates into a hydrogel;
(d) selecting the cell-MC aggregates showing fast cell growth or expressing pluripotency markers;
(e) expanding the selected cell-MC aggregates by adding fresh MCs to the selected cell-MC aggregates; and
(f) characterizing the cell-MC aggregates to determine whether the somatic cells on the MCs have been reprogrammed to iPSCs;
wherein step (a) and step (b) are carried out under continuous agitation; and
wherein the transcription factors comprise Oct4, Sox2, c-Myc, and Klf4.

36. The method of claim 1, wherein the transcription factors comprise Oct4, Sox2 and Klf4, or
wherein the transcription factors consist of Oct4, Sox2 and Klf4, or
wherein the transcription factors comprise Oct4, Sox2 and Klf4 and exclude c-Myc.

37. The method of claim 36, wherein the transcription factors comprise Oct4, Sox2 and Klf4, and further comprise one or more of Nanog, c-Myc, and LIN28.

38. The method of claim 36, wherein the transcription factors comprise Oct4, Sox2 and Klf4 and exclude c-Myc, and further comprise one or more of Nanog and LIN28.

39. The method of claim 1, wherein the transduction is done via an agent selected from the group consisting of virus, protein, plasmids, PiggyBac, and small molecules, or
wherein the transduction is done via a protein that is an Embryonic Stem Cell (ESC)-derived extract protein, or a Cell-Penetrating Peptide selected from the group consisting of a designed peptide, a natural protein-derived peptide, and a chimeric peptide, or
wherein the transduction is done via a plasmid that is an expression plasmid comprising the complementary DNAs (cDNAs) of Oct3/4, Sox2, and Klf4, or
wherein the transduction is done via a small molecule selected from the group consisting of Ascorbic acid, Valproic acid, and Sodium butyrate, or
wherein the transduction is done via a virus selected from a group consisting of respirovirus, lentivirus, retrovirus, and adenovirus, or
wherein the transduction is done via a respirovirus that is Sendai virus.

40. The method of claim 1, wherein the somatic cells are selected from the group consisting of human somatic cells, bovine somatic cells, and avian somatic cells, or
wherein the somatic cells are selected from the group consisting of cells obtained from blood and/or bone marrow, cells obtained from skin biopsy and fibroblasts, or
wherein the somatic cells are cells obtained from blood and/or bone marrow selected from the group consisting of T cells, erythroblasts, peripheral blood mononuclear cells (PBMCs) and hematopoietic stem cells (HSCs), or
wherein the somatic cells are HSCs, or
wherein the somatic cells are cells obtained from skin biopsy selected from the group consisting of human foreskin fibroblasts (HFF), human dermal fibroblasts and human keratinocytes, or
wherein the somatic cells are human lung fibroblasts.

41. The method of claim 1, wherein the MCs are coated by extracellular matrix (ECM), or
wherein the MCs are coated by ECM selected from the group consisting of laminin, vitronectin, fibronectin, heparan sulfate, and collagen.

42. The method of claim 1, wherein the MCs are selected from the group consisting of positively-charged polystyrene MCs, alginate-based MCs, dextran-based MCs, collagen-based MCs, gelatin-based MCs, acrylamide-based MCs, glass-based MCs, and biodegradable MCs, or
wherein the MCs are biodegradable MCs selected from the group consisting of poly-ε-caprolactone (PCL), Poly (lactic acid-co-glycolic acid) (PLGA) and Positively-charged Cytodex 1.

43. The method of claim 42, wherein the size of the MCs is 90-200 μm.

44. The method of claim 1, further comprising:
(c) immobilizing the cell-MC aggregates into a hydrogel;
(d) selecting the cell-MC aggregates showing fast cell growth or expressing pluripotency markers;
(e) expanding the selected cell-MC aggregates by adding fresh MCs to the selected cell-MC aggregates; and
(f) characterizing the cell-MC aggregates to determine whether the somatic cells on the MCs have been reprogrammed to iPSCs.

45. The method of claim 44, wherein:
(i) the hydrogel is an agarose gel;
(ii) the pluripotency marker in step (d) is selected from the group consisting of Tra-1-60 and Tra-1-81;
(iii) the characterization in step (f) is selected from the group consisting of FACS analysis for pluripotency, evaluation of cell growth capacity, evaluation of multi-passage stability, karyotyping, and evaluation of in vitro tri-lineage differentiation;
(iv) the differentiation in step (g) is via formation of Embryoid bodies (EBs)-like cell-MC aggregates;
(v) the differentiation in step (g) is via formation of EBs-like cell-MC aggregates by continuous agitation;
(vi) the step (e) is performed under continuous agitation;
(vii) the step (e) is performed under continuous agitation in a stirred bioreactor system;
(viii) the step (e) is performed under continuous agitation in a stirred bioreactor system comprising controlled dissolved oxygen, temperature, and pH; and/or
(ix) the steps are integrated with an automated machine.

46. The method of claim 25, wherein:
(i) the hydrogel is an agarose gel;
(ii) the pluripotency marker in step (d) is selected from the group consisting of Tra-1-60 and Tra-1-81;
(iii) the characterization in step (f) is selected from the group consisting of FACS analysis for pluripotency, evaluation of cell growth capacity, evaluation of multi-passage stability, karyotyping, and evaluation of in vitro tri-lineage differentiation;
(iv) the differentiation in step (g) is via formation of Embryoid bodies (EBs)-like cell-MC aggregates;
(v) the differentiation in step (g) is via formation of EBs-like cell-MC aggregates by continuous agitation;
(vi) the step (e) is performed under continuous agitation;
(vii) the step (e) is performed under continuous agitation in a stirred bioreactor system;
(viii) the step (e) is performed under continuous agitation in a stirred bioreactor system comprising controlled dissolved oxygen, temperature, and pH; and/or
(ix) the steps are integrated with an automated machine.
